(19) United States
(12) Patent Application Publication
(10) Pub. No.: US 2013/0225442 A1
(43) Pub. Date: Aug. 29, 2013

(54) Lung Cancer Tests

(71) Applicant: Jeffrey A. Borgia, Chicago, IL (US)

(72) Inventor: Jeffrey A. Borgia, Chicago, IL (US)

(73) Assignee: RUSH UNIVERSITY MEDICAL CENTER, Chicago, IL (US)

(21) Appl. No.: 13/880,103

(22) PCT Filed: Oct. 20, 2011
(86) PCT No.: PCT/US11/57110

§ 371 Date: May 16, 2013

Related U.S. Application Data
(60) Provisional application No. 61/394,944, filed on Oct. 20, 2010.

(51) Int. Cl. G01N 33/68; C12Q 1/68

(52) U.S. Cl. CPC G01N 33/6884; C12Q 1/6886
USPC 506/9; 435/6.11; 435/6.12; 435/7.1; 435/7.4; 435/7.92

(57) ABSTRACT

Methods for the diagnosis of lung cancer and more specifically, non-small cell lung cancer are described. An assortment of biomarkers with diagnostic or prognostic value for non-small cell lung cancer (NSCLC) are used to establish a multi-analyte serum test capable of diagnosing patients with lung cancer. A first method assesses an elevated level of expression of one or more nucleic acids, a polypeptide encoded by the nucleic acid or an autoantibody to said polypeptide. An alternative method includes (a) determining whether or not a patient has NSCLC versus a non-NSCLC lung cancer, and (b) classifying the patient as susceptible to specific treatments if the patient has a NSCLC profile and classifying the patient as not susceptible to a specific treatment if the patient does not have a NSCLC profile.

### CROSS-REFERENCE TO RELATED APPLICATIONS

This application is a 35 USC §371 US National Stage filing of International Patent Application No. PCT/US2011/057110 filed on Oct. 20, 2011 claiming priority under the Paris Convention and 35 USC §119(e) to U.S. Provisional Patent Application Ser. No. 61/394,944, filed on Oct. 20, 2010.

### TECHNICAL FIELD

Methods for the diagnosis of lung cancer and more specifically, non-small cell lung cancers are described. An assortment of biomarkers with diagnostic or prognostic value for non-small cell lung cancers (NSCLC) have been combined to establish a multi-analyte serum test capable of identifying patients with non-small cell lung cancers. Practice of this test would be either for diagnosis of lung cancer or stratifying patients as a companion modality for CT-based screening protocols.

### BACKGROUND

Lung cancer remains the second most diagnosed cancer in the United States and the most common cause of cancer mortality, with an estimated 161,000 deaths in 2008. Eighty percent of all lung cancers are non-small cell lung cancers (NSCLCs). While the overall prognosis for patients with lung cancer is poor, with a five-year survival of less than 15%, patients diagnosed with early stage disease have a much more favorable prognosis. Patients with pathological stage I and II disease have five-year survivals of 57-67% and 38-55%, respectively.

Unfortunately, over half of patients with NSCLC present only after metastasis to lymph nodes or distant sites due to its asymptomatic nature at early stages. Therefore, the best prospect for reducing lung cancer mortality remains earlier detection, when surgical outcomes have the best prognosis. A screening tool capable of early stage detection will allow for decreased lung cancer mortality.

While accepted screening programs for breast, colon, prostate and cervical cancer have been developed with subsequent decreases in overall disease mortality, lung cancer screening programs remain in the research realm. There are currently no established methods for screening individuals at high-risk for lung cancer that has been proven to reduce mortality. Therefore, screening for NSCLC is not currently recommended by any major medical association. Without a nationally-defined screening protocol, there is wide variability in the detection and the initiation of treatment for lung cancer. Since the 1950's, numerous screening methods have been evaluated for this purpose, including chest x-ray, sputum cytology, bronchoscopic procedures, low-dose spiral computed tomography, and molecular diagnosis through nucleic acid or protein biomarkers. These modalities have been evaluated both alone and in several combinations. Even though no screening study for lung cancer has proven efficacy in reducing mortality, several of these strategies have improved the understanding of lung cancer progression and allowed for development of potential future screening and treatment modalities. One of the most promising combinations of these methodologies consists of low-dose spiral computed tomography (CT) with a companion serum test.

Chest radiography has been widely employed historically as a preliminary screening tool due to its wide accessibility, relatively low-cost, and ease of use. Radiographs, however, have very low specificity and sensitivity when compared to more contemporary imaging techniques such as CT. Therefore, radiography has had very modest success in diagnosing early-stage disease. Screening trials have demonstrated that chest radiographs fail to detect 60-80% of early-stage lung cancers that were found in the same study by CT. Recent spiral CT advancements have made the method more effective in detecting tumors at a resectable stage than any other modality currently being used for NSCLC.

Despite the promising results obtained from the recent spiral CT studies with an increase in early stage disease seen over historical controls, CT screening has only recently been shown to reduce mortality from NSCLC. That is, data from the National Lung Screening Trial (NLST) demonstrates a 20% reduction in mortality from NSCLC with CT screening of “high risk” patients. However, CT screening protocols have several limitations that may greatly steer its potential implementation. For example, given the relatively high sensitivity of the technique, coupled with its low specificity, many benign lesions appear as questionable, non-calcified nodules. These lesions frequently require serial screening to evaluate for growth or more definite neoplastic traits. The time interval for discerning which lesions are neoplastic via serial CT scans may be a critical period in the progression of NSCLC. Further, computed tomography itself cannot differentiate early aggressive from nonaggressive NSCLC. Therefore, spiral CT is commonly used in combination with a second diagnostic means, such as PET imaging, to attain a more immediate diagnosis and prognosticate patient outcome. However, the cost of combined imaging modalities may be prohibitive for any wide-spread screening programs for early-stage disease and only potentially server individuals in the highest risk strata and not include a bulk of the “at-risk” population. Another method routinely used to discern these questionable nodules is the combination of spiral CT with CT-directed fine needle aspirates or bronchoscopy. However, the anxiety and discomfort associated with these invasive techniques makes them less than ideal for screening asymptomatic patients.

Thus, recent advancements in low-dose spiral CT technology have made great strides towards improving early detection. However, their ability of low-dose spiral CT to reduce mortality from NSCLC has yet to be established. With the relatively high cost of spiral CT, the high rate of false-positives leading to unnecessary biopsy or surgery, and the need for serial measurements to confirm non-neoplastic disease, an addition of an economical test to compliment the CT screening protocol could improve specificity and cost-effectiveness.

There is a clear need in the art for safe, reliable and simple methods for screening patients for the presence and stage of NSCLC and for the discrimination between benign pulmonary disorders.

### SUMMARY OF THE DISCLOSURE

In one example, nucleic acids and/or their polypeptide products and/or autoantibodies to their polypeptide products, are used as lung cancer biomarkers, having variant over-expression in tumors. Such nucleic acids, as well as polypeptides encoded by such nucleic acids and autoantibodies against these polypeptides, can be analyzed to assess lung cancer and more particularly, NSCLC, in mammals. Analysis of the nucleic acids, or polypeptides encoded by the nucleic acids, may allow lung cancer to be assessed in mammals based on an elevated level of one or more of the nucleic acids or polypeptides in a biological sample (e.g., a biopsy specimen) from the mammal. The levels of multiple nucleic acids or polypeptides may be detected simultaneously using nucleic acid or polypeptide arrays.

A low-cost and minimally invasive serum, sputum or tissue test is a much preferred means to complement spiral CT or potentially serve as a pre-screening method to minimize the overall costs of NSCLC detection. No FDA-approved test of this sort currently exists.

In one aspect, a method for assessing lung cancer is provided. The method may comprise determining whether or not a mammal having lung cancer comprises an elevated level of expression of one or more nucleic acids, or a polypeptide encoded by the nucleic acid or an autoantibody to said polypeptide, selected from the group consisting of TNF-α, CYFRA 21.1, IL-1ra, IL-6, IFN-γ, IL-2Rα, CA125, MCP-1, CRP, MMP-2 and sE-selectin where the presence or absence of the elevated level indicates that the mammal is susceptible to a poor outcome. The method may comprise determining whether or not a mammal having lung cancer comprises an elevated level of an TNF-α, CYFRA 21.1, IL-1ra, IL-6, IFN-γ, IL-2Rα, CA125, MCP-1, CRP, MMP-2 and sE-selectin nucleic acid, or a polypeptide encoded by the TNF-α, CYFRA 21.1, IL-1ra, IL-6, IFN-γ, IL-2Rα, CA125, MCP-1, CRP, MMP-2 and sE-selectin nucleic acid or an autoantibody to the polypeptide encoded b the TNF-α, CYFRA 21.1, IL-1ra, IL-6, IFN-γ, IL-2Rα, CA125, MCP-1, CRP, MMP-2 and sE-selectin nucleic acid. The mammal may be a human. The level may be determined in lung tissue, sputum or blood. The level may be determined using PCR or in situ hybridization. The level may be determined using immunohistochemistry. The poor outcome may comprise systemic progression within five years of drug or radiation treatment.

In one embodiment, the method may comprise determining whether or not a mammal has lung cancer such as NSCLC which the method comprises detecting an elevated level of one or more nucleic acids, polypeptides or autoantibodies selected from the group consisting of TNF-α, CYFRA 21.1, IL-1ra, MMP-2, MCP-1, and sE-selectin. In a further preferred embodiment, the method may comprise determining the level of one or more nucleic acids, polypeptides or autoantibodies selected from the group consisting of TNF-α, CYFRA 21.1, IL-1ra, MMP-2, MCP-1, and sE-selectin and using the presence or absence of an elevated level to determine treatment for lung cancer such as NSCLC.

In an alternate use, the serum, tissue or autoantibody tests described above may be used as an initial screen to assess NSCLC risk, and select for a smaller population that requires further screening with spiral CT.

In another aspect, a method for assessing lung cancer is provided. The method may comprise: (a) determining whether or not a mammal has NSCLC versus a non-NSCLC lung cancer, and (b) classifying the mammal as susceptible to specific treatments if the mammal has a NSCLC profile and classifying the mammal is not susceptible to a specific treatment if the mammal does not have a NSCLC profile.

The method for differentiating NSCLC versus benign lung diseases, may comprise determining whether or not a mammal has an elevated level of expression of one or more nucleic acids, or a polypeptide encoded by the nucleic acid or an autoantibody to said polypeptide, selected from the group consisting of inosine-5-monophosphate dehydrogenase (IMPDH), fumarate hydratase (FH), α-enolase, endoplasmic reticulum protein 29 (Erp29), annexin I, hydrosteroid 17-β dehydrogenase, methylthioadenosine phosphorylase (MTAP), annexin II, ubiquilin, c-Myc, NY-ESO, 3-oxoacid CoA transferase, p53 phosphoglycerate mutase, and heat shock protein 70-9B (HSP70-9B)]. The mammal may be a human. The NSCLC profile may be determined in lung biopsy tissue, sputum, urine, or blood. The NSCLC profile may be determined using PCR or a nucleic acid array. The NSCLC profile may be determined using immunohistochemistry or an array for detecting polypeptides. The determination of treatment regimen and outcome may be based on comparison of a newly determined profile of a patient against a statistical analysis of a sample population of patients treated via specific therapeutic regimens versus outcome (including but not limited to five year survival) versus NSCLC profiles for the sample population.

In another embodiment, the method may comprise determining whether or not a mammal having lung cancer versus a nonmalignant disease may comprise detecting an elevated level of one or more nucleic acids, polypeptides or autoantibodies selected from the group consisting of IMPDH, phosphoglycerate mutase, ubiquillin, annexin I, annexin II, and heat shock protein 70-9B (HSP70-9B). In a further preferred embodiment, the method may comprise determining the level of one or more nucleic acids, polypeptides or autoantibodies selected from the group consisting of IMPDH, phosphoglycerate mutase, ubiquillin, annexin I, annexin II, and heat shock protein 70-9B (HSP70-9B)] and using the presence or absence of an elevated level to determine treatment for lung cancer such as NSCLC.

Alternately, a serum, tissue or autoantibody test may be used to discriminate between non-neoplastic disease and malignancy for a questionable nodule found by CT, thereby eliminating the need for serial CTs or invasive biopsy. In this alternative use, the clinically-useful multi-analyte nucleic acid, polypeptide or autoantibody panel described above can discern NSCLC from “non-NSCLC” populations for a questionable nodule found by CT, thereby eliminating the need for serial CTs or invasive biopsy. A prognostic application may help guide adjuvant treatment strategies.

### BRIEF DESCRIPTION OF THE DRAWINGS

FIGS. 1A-1C show representative 2D protein gel of HCC827 cellular lysates with accompanying immunoblots. Coomassie stained 2D gel for proteins isolated from HCC827 cellular lysates (A); with matched immunoblots from the control (B) and stage I adenocarcinoma (C) cohorts.

FIGS. 2A-2F show individual ‘box and whisker’ plots for six selected biomarkers and distributions of circulating autoantibody levels with cohorts separated as follows: 1—NSCLC patients; 2—COPD/asthma patients; 3—“cancer-free” control patients; and 4—resected patients with non-neoplastic nodules. Abbreviations: MFI-scaled—median fluorescent intensity values scaled to a standard concentration of appropriate commercial antibody; PGAM—phosphoglycerate mutase; and IMPDH—inosine monophosphate dehydrogenase.

FIG. 2G-2L are box and whisker plots for six biomarkers identified by the Random Forest algorithm. Box plots for the six selected biomarkers were selected by the Random Forest analysis on the discovery cohort. Abscissa labels: 0=surgically resected, non-neoplastic nodules; 1=‘normal’ controls; 2=Stage 1A NSCLC; 3=Stage 1B NSCLC; and 4=Stages 1I and III (node positive) NSCLC. Notes: disease staging is based on pathologic stage; extreme values are not shown in the plots. Significance (Mann-Whitney Rank sum test) is shown with bars above boxes with a=P<0.001; b=P<0.01 and c=P<0.05.

FIG. 3A is a classification and regression tree (CART) based algorithm for the six autoantibody panel of FIGS. 2A-2F. Briefly, the algorithm represents a series of binary ‘if-then’ decision rules that are used to split the data into separate branches of the tree. Each node of the tree displays the analyte being considered and the threshold concentrations used to partition the patient groups. Additional classifications continue along each arm of the split where it is indicated whether the measured value is either less than or equal to or exceeding the indicated threshold cutoff value. The diagnosis by this decision tree is listed at each terminal node, with each final arm on the left indicating cases predicted as “Benign” and each final arm on the right predicted as “NSCLC”. Abbreviations: MFI—median fluorescent intensity values scaled to a standard concentration of appropriate commercial antibody; PGAM—phosphoglycerate mutase; HSP70-9B—heat shock protein 70 kDa protein 9B (mortalin-2); and IMPDH—inosine monophosphate dehydrogenase.

FIG. 3B is another classification and regression tree for the six analytes of FIGS. 2G-2L. The classification and regression tree of FIG. 3B is for predicting whether a patient is positive for NSCLC. Briefly, the algorithm represents a series of binary ‘if-then’ decision rules that are used to split the data into separate branches of the tree. Each node of the tree displays the analyte being considered and the threshold concentrations used to partition the patient groups. Additional classifications continue along each arm of the split in which it is indicated whether the measured value is either less than or equal to or exceeding the indicated threshold cutoff value. The number of classifications (observations) are listed immediately below each terminal node, with each final arm labeled (0=NSCLC negative; 1=NSCLC positive). Abbreviations: obs.=observations; TNF-a=tumour necrosing factor-a; MCP-1=monocyte chemotactic protein-1; MMP-2=matrix metalloproteinase-2 and IL-1ra=interleukin-1 receptor antagonist.

FIG. 4 is an ROC curve for the six analyte serum test using the six analytes of FIGS. 2G-2L and FIG. 3B. The ROC curve is for the optimized six-analyte CART algorithm using the original training cohort of patients. The area under the curve is 0.979, the sensitivity is 99% and the specificity is 95%.

FIG. 5 illustrates a general approach for serum biomarker discovery. This figure illustrates the general steps in our overall candidate biomarker discovery strategy. The approach proposed will examine six patient groups and a control reference-peptide group; space considerations limited the experiment to four groups. (RFS—recurrence-free survival).

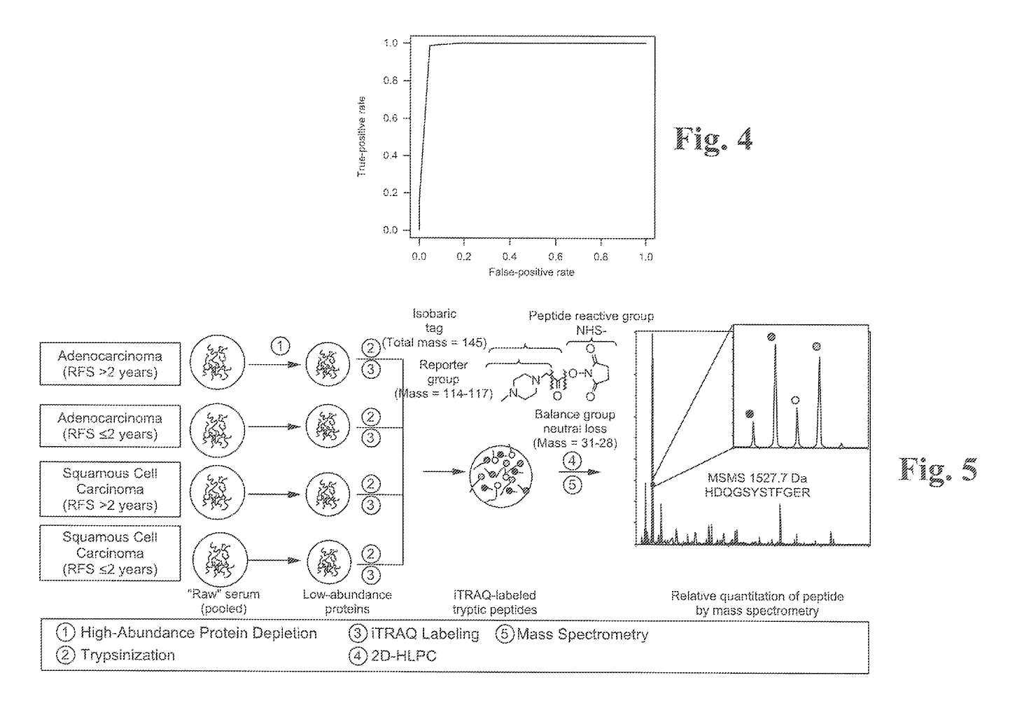

### DETAILED DESCRIPTION

A serum test is disclosed that may be used as an initial screen to assess lung cancer risk, and select for a smaller population that requires further screening with spiral CT. Alternately, a serum test is used to discriminate between non-neoplastic lung disease and lung cancer malignancy for a questionable nodule found by CT, thereby eliminating the need for serial CTs or invasive biopsy. In a preferred embodiment the lung cancer is NSCLC. In a further alternative, a tissue or autoantibody test can be used to screen a patient for lung cancer risk or to discriminate between non-neoplastic disease and malignancy. The test can screen for nucleic acids encoding one or more specific biomarkers, the polypeptides transcribed by those biomarker nucleic acids, or autoantibodies to the polypeptides transcribed by those biomarker nucleic acids in order to screen a patient for lung cancer risk or to discriminate between non-neoplastic disease and malignancy. The test may also provide a prognosticating outcome or detect the presence of metastatic disease.

In a first example, an array of 47 candidate biomarkers implicated in NSCLC was selected and screened a total of 135 patients (n=92 NSCLC; n=43 ‘healthy’ control) to evaluate the panel of biomarkers with significant test performance characteristics for differentiating between patients with early-stage NSCLC and our control population. From the selected biomarkers, a multi-analyte blood test capable of screening for NSCLC either as a stand-alone diagnostic measure or as a companion test for current CT-based screening protocols was identified.

Serum specimens were obtained from 92 NSCLC patients as well as two different groups of controls (n=43) to approach the complexity that “high risk” populations pose to a diagnostic measure of this type. All NSCLC patients and controls were obtained in full compliance with the Institutional Review Board including formal written consent. Diagnosis confirmation for the NSCLC cohort was obtained from surgical pathology reports on tissue gathered from tumor resection with lymph node dissections. Criteria for study inclusion in the NSCLC cohort were broad (consisted of having a surgical resection with pathological evaluation) and were not limited to any demographic or clinical factor. ‘Normal’ control specimens (n=31) were obtained. This cohort was selected on the basis of similar demographic characteristics (with respects to age and sex) and had a diagnosed condition with an inflammatory component. Seven of 31 patients in this cohort had a significant smoking history. At the time of specimen accrual, and in clinical follow-up data, these patients had no evidence of any pulmonary disorders or carcinomas of any type. The ‘non-neoplastic disease after surgery’ group consisted of 12 patients with granulomas, pneumonitis or pneumonia. These patients underwent resection secondary to concern for cancer or persistent symptoms after conservative management.

The specimens used for panel validation consisted of the following cohorts: a NSCLC cohort (n=33 total) consisting of 25 stage I, seven stage II and one stage III NSCLC patients. A second control cohort of 15 non-neoplastic lung disease patients with surgically resected “questionable” lesions and a ‘non-neoplastic disease without surgery’ group consisting of 40 patients with chronic obstructive pulmonary disease (COPD) or asthma were also used in the validation studies. Patients from this COPD/asthma group were seen clinically based on complaints of cough development or change in respiratory symptoms; serum was collected immediately preceding bronchoscopy and CT imaging was then used to evaluate for the presence of pulmonary nodules. The overall COPD/asthma cohort from which these cases were selected possessed a smoking history similar to the NSCLC cohort (median value of 40 pack years). Peripheral blood was collected from each patient immediately prior to treatment initiation for NSCLC. Ten mL of blood was drawn into standard red-top Vacutainers® (without anticoagulants) and coagulated at room temperature for 30-40 min. Sera was then separated with centrifugation. Yields ranged 4-7 mL of serum per 10 mL of whole blood. Sera was then immediately divided into aliquots and archived in an −80° C. ultra-low temperature freezer. No specimens were subjected to more than two thaw cycles for this study.

Peripheral blood was obtained from each patient immediately prior to treatment initiation using standard phlebotomy techniques, with all samples handled and processed in an identical manner, as described above. No specimens were subjected to more than two thaw cycles for this study. Control sera were collected in an identical manner and processed as described above.

Whenever possible, the Luminex xMAP® immunoassay platform was used to measure the circulating levels of biomarkers, with ELISA-based immunoassays encompassing only two of the 47 biomarkers tested. All assays were performed according to the respective manufacturer's instructions and were conducted in the following groupings: C-reactive protein (CRP) and serum amyloid A (SAA) [Millipore; Billerica, Mass.]; Interleukin-1β (IL-1β), IL-1ra, IL-6, IL-8, IL-10, tumor necrosis factor-α (TNF-α), and transforming growth factor-α (TGF-α) [Millipore; Billerica, Mass.]; IL-2, IL-13, interferon-γ (IFN-γ), interferon-inducible protein 10 (IP-10), and granulocyte monocyte colony stimulating factor (GM-CSF) [Bio-Rad Laboratories; Hercules, Calif.]; IL-1α, IL-2Rα, M-CSF, stem cell-derived factor 1α (SDF-1α), and stem cell factor (SCF) [Bio-Rad Laboratories; Hercules, Calif.]; sE-selectin, sP-selectin, and soluble intracellular adhesion molecule 1 (sICAM 1) [R & D systems; Minneapolis, Minn.]; matrix metalloproteinase-2 (MMP-2), MMP-3, MMP-9, and MMP-13 [R & D Systems; Minneapolis, Minn.]; death receptor 5 (DR-5), tissue necrosis factor—receptor I (TNF-RI), and TNF-RII [Invitrogen; Carlsbad, Calif.]; RANTES, macrophage inflammatory protein-1α (MIP-1α), MIP-1β, monocyte chemotactic protein-1 (MCP-1), and eotaxin [Invitrogen; Carlsbad, Calif.]; granulocyte colony stimulating factor (G-CSF), epidermal growth factor (EGF), vascular endothelial growth factor (VEGF), and basic fibroblast growth factor (bFGF) [Invitrogen; Carlsbad, Calif.]; and sEGFR (erb-bl), Her-2 (erb-b2), CA125, CA15-3, CA19-9, CEA, and CYFRA 21.1 were measured at the University of Pittsburgh Cancer Institute's Luminex Core Facility. Biomarker concentrations were calculated through a five-parametric curve fit as part of the BioPlex Suspension Array System Software v4.0 (Bio-Rad Laboratories; Hercules, Calif.). Measurements of TIMP-1 and osteopontin concentrations were conducted using commercially-available ELISA assays and in accordance to the kit directions (R & D Systems; Minneapolis, Minn.). Data was collected on a BioTek PowerWave XS plate reader using KC Junior (v1.40.3) software package. A four-parametric curve fit was used to calculate the concentrations from the raw absorbance readings. All assays performed for this study were conducted in a blinded fashion and were statistically processed by different personnel to minimize operator bias.

Validation studies used the identical commercially available kits for 14 of the analytes evaluated, following manufacturer's instructions in the following groupings: CRP [Millipore; Billerica, Mass.]; IL-1ra, IL-6, IL-10, and TNF-α [Millipore; Billerica, Mass.]; interferon-γ (IFN-γ) [Bio-Rad Laboratories; Hercules, Calif.]; IL-2Rα [Bio-Rad Laboratories; Hercules, Calif.]; sE-selectin and sP-selectin [R & D systems; Minneapolis, Minn.]; MMP-2 [R & D Systems; Minneapolis, Minn.]; MIP-1α, MCP-1, and eotaxin [Invitrogen; Carlsbad, Calif.]; CA125 and CYFRA 21.1 were measured at the University of Pittsburgh Cancer Institute's Luminex Core Facility. The data was collected in the same manner and a 5-parametric curve fit was used to calculate the concentrations from the raw absorbance readings.

The initial selection consisted of an array of 47 biomarkers; they were selected based either on published reports for each biomarker showing value for at least one of the following functions: NSCLC diagnosis, staging, or prognosis or involvement in biological processes implicated in disease progression. The levels of these markers were evaluated in sera from 92 NSCLC patients and 43 non-cancer controls. Tables 1-3 show the clinical and pathological characteristics of patients.

Several biomarkers, including IL-1α, IL-1β, IL-2, IL-15, GM-CSF, TGF-α, DRS, MMP-13, had a significant portion of their measurements fall below the threshold of assay range and were disqualified from further analysis. These biomarkers exhibited no apparent trends in the raw data warranting reanalysis.

TABLE 1
Basic characteristics of groups defined
for biomarker discovery/validation.

| | p | | | Mini- | Maxi- |
|---|---|---|---|---|---|
| Biomarker | value | AUC | Median | mum | mum |
| IGF BP5 | 0.00024 | 0.744 | 229.73 | 2.515 | 10573.625 |
| p53 | 0.0102 | 0.672 | 2106.3 | 231 | 20598 |
| protein disulfide | 0.01734 | 0.656 | 514.05 | 73.16 | 4000 |
| isomerase 3 | | | | | |
| heat shock | 0.01856 | 0.662 | 897.44 | 215.24 | 4000 |
| protein 5a | | | | | |
| Recoverin | 0.02679 | 0.632 | 1979.8 | 174.3 | 18603.5 |
| Peroxiredoxin | 0.03298 | 0.628 | 1895 | 234 | 19334 |
| stem cell factor | 0.04354 | 0.637 | 50.85 | 13.18 | 246.44 |
| NY-ESO | 0.05285 | 0.62 | 2226.75 | 183.5 | 20353.25 |
| Alpha enolase | 0.05684 | 0.616 | 2033 | 561 | 13339 |
| IGF 1 | 0.06461 | 0.588 | 46.1621 | 0.49122 | 586.54402 |
| glyceraldehyde-3- | 0.06467 | 0.617 | 4059 | 925 | 16595 |
| phosphate DH | | | | | |
| triosephosphate | 0.06842 | 0.598 | 1948.8 | 221.5 | 21755 |
| isomerase | | | | | |
| 3-oxoacid CoA | 0.07438 | 0.608 | 1191.25 | 302.75 | 12969.25 |
| transferase | | | | | |
| methylthio- | 0.07646 | 0.604 | 1171 | 317.25 | 11972.75 |
| adenosine | | | | | |
| phosphorylase | | | | | |
| Ubiquilin | 0.07858 | 0.608 | 876 | 220 | 11739 |
| Hydroxyacyl- | 0.08522 | 0.606 | 384.07 | 70.18 | 4000 |
| CoA | | | | | |
| dehydrogenase | | | | | |
| Surviving | 0.0864 | 0.618 | 2503 | 596 | 11243 |
| isocitrate | 0.09478 | 0.606 | 483.85 | 128.94 | 4000 |
| dehydrogenase | | | | | |
| heat shock | 0.09994 | 0.624 | 691.97 | 119.88 | 4000 |
| protein 9a | | | | | |
| annexin A1 | 0.146 | 0.574 | 328.8000 | 49.46 | 4003.16 |

TABLE 2
Characteristics of Patient Populations

| | Discovery | | | | | | Validation | | | | | |
|---|---|---|---|---|---|---|---|---|---|---|---|---|
| | I | | II | | III | | I | | II | | III | |
| Age | | | | | | | | | | | | |
| Range | 40 | 83 | 47 | 80 | 46 | 84 | 40 | 80 | 49 | 92 | 46 | 84 |

| | | | | | | |
|---|---|---|---|---|---|---|
| Median | 67.5 | 62 | 69 | 61 | 70 | 68 |
| Sex | | | | | | |
| Male | 3 | 9 | 38 | 6 | 21 | 17 |
| Female | 9 | 22 | 52 | 9 | 19 | 16 |
| NSCLC stage^{a} | | | | | | |
| Ia | | | 16 | | | 11 |
| Ib | | | 37 | | | 14 |
| IIa | | | 2 | | | 2 |
| IIb | | | 11 | | | 5 |
| IIIa | | | 19 | | | 1 |
| IIIb | | | 5 | | | |
| Diagnosis | | | | | | |
| Adenocar- | | | 57 | | | 18 |
| cinoma | | | | | | |
| Squamous | | | 30 | | | 10 |
| Adeno- | | | 1 | | | 2 |
| squamous | | | | | | |
| NSCLC other | | | 2 | | | 3 |
| Condition | | | | | | |
| No lung | | 31 | | | | |
| pathology | | | | | | |
| Granuloma | 5 | | | 6 | | |
| COPD | | | | 1 | 34 | |
| Asthma | | | | | 6 | |
| Sarcoidosis | 1 | | | | | |
| Pneumonitis | 1 | | | 1 | | |
| Pneumonia | 2 | | | 1 | | |
| Benign cyst. | 1 | | | | | |
| Hamartoma | | | | 2 | | |
| Chronic | | | | 2 | | |
| inflammation | | | | | | |
| Lymphoid | | | | 1 | | |
| infiltrate | | | | | | |
| Thymoma | 1 | | | | | |
| Lipoma | 1 | | | 1 | | |

Discovery group refers to the initial group of patients on which 47 biomarkers were tested and multi-analyte panel was created, groups are as follows: I resected non-neoplastic disease; II rheumatology controls; III NSCLC patients.
Validation group refers to second cohort on whom our six-multi-analyte panel was tested, groups are as follows: I resected non-neoplastic disease; II COPD/asthma patients; III NSCLC patients.
^{a}Pathologic stage.

Two more patient groups were enrolled in the “discovery” portion of this study, as defined in Table 3. The first group consisted of 10 patients with lung adenocarcinoma (T1-2N0M0) that received complete anatomic resections with curative intent at Rush University Medical Center (RUMC), Chicago, Ill. The second group that served as a control for the study was comprised of patients (n=10) with non-neoplastic pulmonary disorders that were resected at RUMC under suspicion of having NSCLC.

TABLE 3
Characteristics of Patient Populations

| | Discovery | | Validation | | | |
|---|---|---|---|---|---|---|
| | control | AdC | 1 | 2 | 3 | 4 |
| Age | | | | | | |
| Range | 56-83 | 65-86 | 46-85 | 49-92 | 47-80 | 39-84 |
| Mean | 69 | 72 | 68 | 68 | 61 | 63 |
| Sex | | | | | | |
| Male | 7 | 6 | 52 | 16 | 8 | 5 |
| Female | 3 | 4 | 65 | 16 | 23 | 11 |
| NSCLC | | | | | | |
| Ia | | 2 | 28 | | | |
| Ib | | 7 | 49 | | | |
| IIa | | 1 | 2 | | | |
| IIb | | | 13 | | | |
| IIIa | | | 17 | | | |
| IIIb | | | 4 | | | |
| IV | | | 4 | | | |
| Diagnosis | | | | | | |
| Adenocarcinoma | | 10 | 63 | | | |
| Adenosquamous | | | 3 | | | |
| Squamous | | | 33 | | | |
| Large Cell | | | 2 | | | |
| NSCLC-NOS | | | 16 | | | |
| Condition | | | | | | |
| Asthma | | | | 6 | | |
| COPD | | | | 26 | | |
| Osteoarthritis | | | | | 31 | |
| Granuloma | 6 | | | | | 8 |
| Pneumonia | 3 | | | | | 4 |
| Benign cyst | 1 | | | | | 1 |
| Sarcoma | | | | | | 1 |
| Lipoma | | | | | | 2 |

Serum concentrations of TNF-α, CYFRA 21.1, IL-1ra, IL-6, IFN-γ, IL-2Rα, and CA125, were found to be significantly higher in the NSCLC group (Mann-Whitney rank sum (two-sided) p-values less than or equal to 0.001) whereas the concentration of MCP-1, CRP, MMP-2 and sE-selectin were found to be significantly higher in the control group (p-values less than or equal to 0.001). Using a significance threshold of a Mann-Whitney rank sum (two-sided) p value less than 0.05 or analysis of the ROC curve ‘area under the curve’ (AUC) greater than 0.65, a total of 14 biomarkers were found to be suitable to undergo multivariate analysis. A list of these biomarkers along with the statistical parameters for each is included in Tables 4-6. No significant differences were observed upon examination of biomarker levels associations with age, smoking history, and fasting status (all p-values were >0.1).

Serum specimens from four patient populations (n=196) were included in the Luminex validation study. See Table 3 for the basic patient demographics of these groups. The NSCLC group consists of 81 patients with early-stage (T1-3N0M0) disease, 32 with locally-advanced disease (T1-3N1-2M0), and 4 with distant metastases. Criteria for study inclusion in the NSCLC cohort were broad (consisted of having a surgical resection and/or pathological confirmation of NSCLC) and were not limited to any demographic or clinical factor. Patients treated with neoadjuvant chemo- or radiation therapy were excluded from the study. All patients in the NSCLC cohort underwent definitive resection of their primary tumor with systematic lymph node dissection. All serum specimens from patients receiving anatomic resections were collected immediately prior to surgery and were, therefore, fasting.

The control cohort consisted of three separate groups: 31 volunteers seen in our Rheumatology Department as part of an osteoarthritis study (known hereafter as the “osteoarthritis” cohort) and had no history of lung disease or carcinomas of any type at the time of serum collection or in 3 years of clinical follow-up; de-identified COPD and asthma patients (n=32 total) from Abbott Laboratories (North Chicago, Ill.); and 16 patients that received anatomic resections at RUMC for suspected NSCLC that, upon pathological diagnosis, were diagnosed with non-neoplastic nodules (caseating granulomas, resolving pneumonias, etc). The Abbott cohort was collected at the time of a bronchoscopy procedure (performed to investigate symptom escalation) and was fasting at the time of serum accrual. Patients participating in the osteoarthritis study had their serum collected as part of a normal office visit and no requirement for fasting was made as part of this protocol.

A panel of six biomarkers was selected from the 14 biomarkers meeting our inclusion criteria for statistical relevance using the Random Forests algorithm, as defined in the materials and methods section. The averaged out-of-bag ‘misclassification errors’ as well as the AUC from the range of the 1000 trees of the Random Forest grown for each of their respective sub-panels are shown in Tables 4-6:

TABLE 4
Biomarker List and Characteristics

| | NSCLC: Node | | NSCLC: Node | | | |
|---|---|---|---|---|---|---|
| | Negative^{‡} | | Positive^{‡} | | | |
| | (n = 71) | | (n = 36) | | | Mann- |
| Biomarker | Median* | Range* | Median* | Range* | AUC | Whitney U |
| MIP-1α | 0.13 | 0.09-0.70 | 0.12 | 0.05-0.81 | 0.775 | 0.081 |
| SCF | 52.8 | 13.2-246.4 | 46.5 | 16.7-110.4 | 0.715 | 0.011 |
| TNF-α | 14.5 | 4.5-55.0 | 13.7 | 2.9-41.9 | 0.689 | 0.141 |
| IL-2R_{α} | 45.2 | 2.1-452 | 47.0 | 7.0-192.8 | 0.688 | 0.598 |
| TNF-RII | 1.87 | 0.32-7.87 | 1.60 | 0.22-4.42 | 0.684 | 0.223 |
| M-CSF | 2.27 | 0.05-34.7 | 1.28 | 0.05-12.0 | 0.679 | 0.041 |
| sICAM-1 | 9,608 | 5,148-17,860 | 10,128 | 5,058-28,376 | 0.675 | 0.408 |
| CEA | 1,392 | 255-54,741 | 1,717 | 255-32,506 | 0.660 | 0.191 |
| G-CSF | 0.117 | 0.014-0.070 | 0.150 | 0.014-0.580 | 0.657 | 0.247 |
| IFN-γ | 33.2 | 5.7-2,258 | 8.2 | 5.7-1,417 | 0.629 | 0.011 |
| TNF-RI | 1.69 | 0.62-8.14 | 1.41 | 0.57-8.13 | 0.623 | 0.546 |
| Osteopontin | 25.3 | 0-195 | 22.5 | 0-190.0 | 0.616 | 0.640 |
| IL-1ra | 290.7 | 1.2-5,796 | 321.7 | 1.1-3,258 | 0.600 | 0.898 |
| CRP | 212.6 | 1.5-1,018.5 | 293 | 1.5-992.2 | 0.576 | 0.027 |
| MMP-2 | 6006 | 3,293-12,536 | 5,589 | 897-10,094 | 0.568 | 0.118 |

*values expressed as pg/mL
^{‡}Based on pathologic staging.

TABLE 5
Biomarkers selected for Multivariate Analysis based on Statistical Relevance

| | Resected benign | | | ‘Normal’ | | | NSCLCₜ | | | NSCLCₜ | | | Overall performance | |
|---|---|---|---|---|---|---|---|---|---|---|---|---|---|---|
| | nodules (n 12) | | | controls (n 31) | | | Stage I^{a} (n 55) | | | Stages II &III^{a,b} (n 37) | | | characteristics | |
| Biomarker | Median^{c} | Range^{c} | | Median^{c} | Range^{c} | | Median^{c} | Range^{c} | | Median^{c} | Range^{c} | | AUC | P-value^{d} |
| CYFRA 21-1 | 371 9 | 227 | 783 | 277.54 | 1223 | 617.2 | 630.89 | 169.5 | 5351 | 674.56 | 293.4 | 3663 | 0.873 | <0.001 |
| TNF-α | 8.92 | 3.7 | 27 | 4.06 | 1.37 | 11.94 | 14.35 | 1.37 | 1.40 | 13.85 | 2.9 | 41.93 | 0.862 | <0.001 |
| MCP-1 | 0.47 | 0.28 | 1.07 | 0.77 | 0.2 | 2.34 | 0.40 | 0.18 | 2.06 | 0.4 | 0.12 | 5.7 | 0.753 | <0.001 |
| IL-1ra | 184.2 | 42 | 1168 | 81.59 | 2.41 | 606.4 | 326.93 | 2.41 | 2144 | 326.93 | 1.12 | 3258 | 0.719 | <0.001 |
| MMP-2 | 679.2 | 4440 | 10013 | 7757 | 5029 | 12836 | 6195 | 3293 | 12693 | 5516 | 897 | 10094 | 0.705 | <0.001 |
| IL-6 | 37.59 | 1.17 | 1495 | 11.39 | 1.17 | 1520 | 61.46 | 1.17 | 5862 | 54.99 | 3.44 | 906.5 | 0.702 | <0.001 |
| EDTAXIN | 0.135 | 0.07 | 0.23 | 0.16 | 0.06 | 0.26 | 0.10 | 0.04 | 0.39 | 0.11 | 0.04 | 0.56 | 0.698 | <0.001 |
| CA-125 | 1.13 | 0.43 | 11.47 | 0.57 | 0.12 | 4.92 | 1.38 | 0.12 | 143.4 | 1.8 | 0.1 | 24.6 | 0.698 | <0.001 |
| sE-selectin | 1643 | 702 | 3962 | 1635 | 862 | 4079 | 1198 | 417 | 2603 | 1283 | 509 | 2547 | 0.690 | <0.001 |
| sP-selectin | 2779 | 1265 | 8764 | 3168 | 17.22 | 51.58 | 2726 | 9.26 | 5181 | 1892 | 926 | 11835 | 0.677 | <0.001 |
| MIP-1α | 0.13 | 0.1 | 0.15 | 0.14 | 0.12 | 0.21 | 0.13 | 0.1 | 0.7 | 0.12 | 0.05 | 0.81 | 0.669 | 0.00117 |
| IL-10 | 21.32 | 3.04 | 92.08 | 3.04 | 3.04 | 576.6 | 16.83 | 3.04 | 1361 | 42.5 | 3.04 | 428 | 0.667 | 0.00162 |
| CRP | 2659 | 0.945 | 4.336 | 4.89 | 0.945 | 23.7 | 2.69 | 0.95 | 10.2 | 3.05 | 0.015 | 9.92 | 0.662 | 0.00245 |
| IL-2Rα | 54.4 | 18.7 | 119.7 | 29.815 | 5.33 | 224.62 | 57.44 | 5.33 | 3359 | 46.98 | 7.03 | 192.8 | 0.652 | 0.00462 |

^{a}Pathologic stage.
^{b}Lymph node-positive disease.
^{c}Values expressed as pgml¹.
^{d}Mann Whitney U (two-sided test). Description statistical parameters and individual test performance characheristics measured from our training cohort for each biomarker within our statistical thresholds.
Abbreviations:
NSCLC non-small cell lung cancer.
AUC area under the curve;
TNF tumors necrosis factor;
MCP monocyte chemotactic protein;
IL interleukin;
MMP matrix metalloproteinases;
CA cancer antigen;
MIP macrophage inflammatory protein;
CRP C-reactive protein;
CYFRA CYFRA 21-1, cytoloeratin 19 fragment.

TABLE 6
Biomarker Panel and Selection Criteria

| | | Biomarkers | | | | | | | | | | | | | | | |
|---|---|---|---|---|---|---|---|---|---|---|---|---|---|---|---|---|---|
| | | MIP- | | | TNF- | TNF- | | M- | G- | | | | | | Osteo- | | |
| | Variables | 1α | SCF | CEA | RI | α | IFN-γ | CSF | CSF | TNF-RII | sICAM-1 | MMP-2 | CRP | IL-2Rα | pontin | IL-1ra | OOB |
| 1 | 15 | X | X | X | X | X | X | X | X | X | X | X | X | X | X | X | 0.336 |
| 2 | 12 | X | X | X | X | X | X | X | X | X | X | X | X | | | | 0.308 |
| 3 | 10 | X | X | X | X | X | X | X | X | X | X | | | | | | 0.308 |
| 4 | 8 | X | X | X | X | X | X | X | X | | | | | | | | 0.317 |
| 5 | 6 | X | X | X | X | X | X | | | | | | | | | | 0.308 |
| 6 | 5 | X | X | X | X | X | | | | | | | | | | | 0.289 |
| 7 | 4 | X | X | X | X | | | | | | | | | | | | 0.345 |

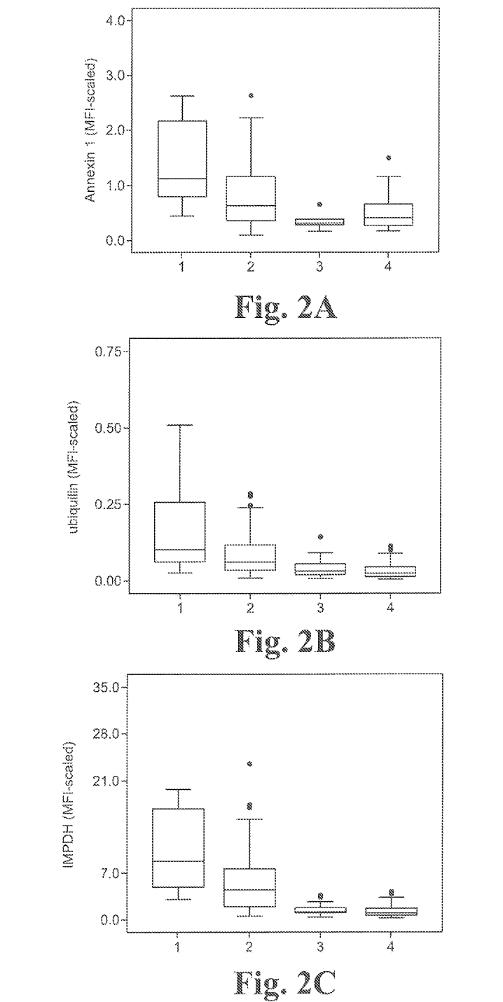

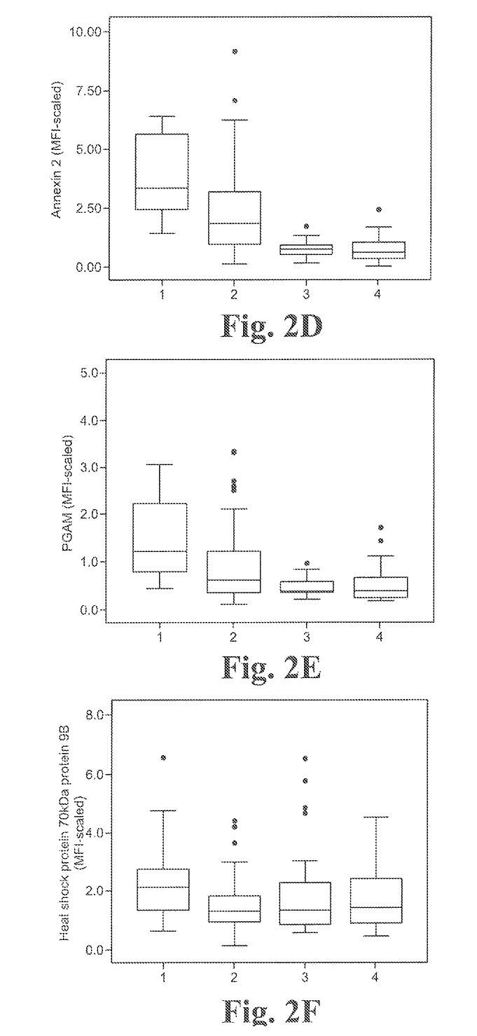

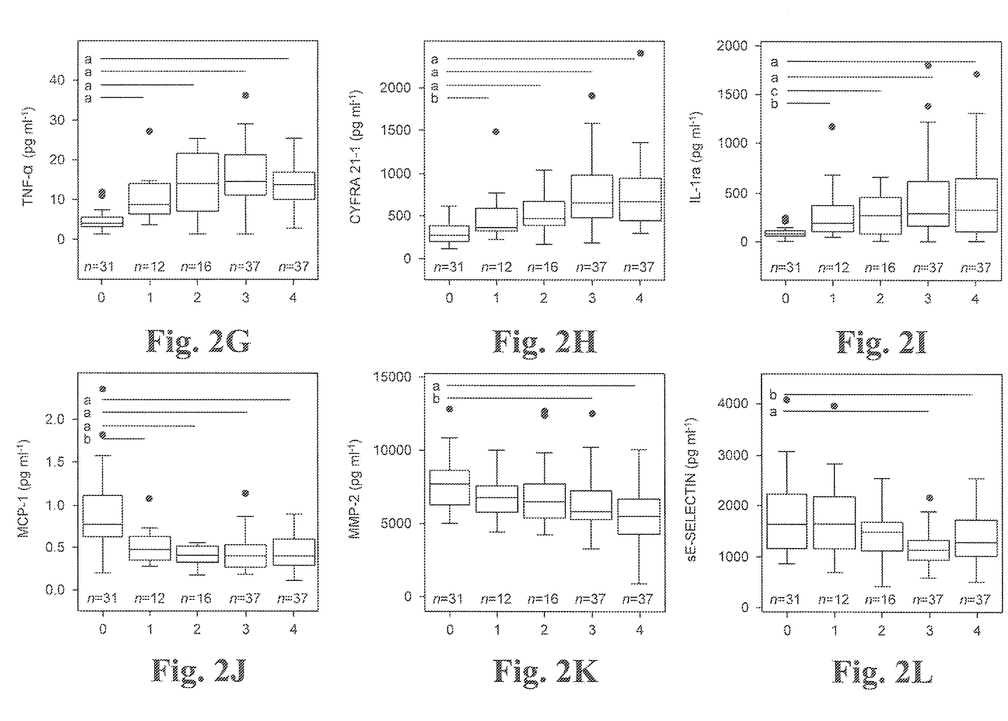

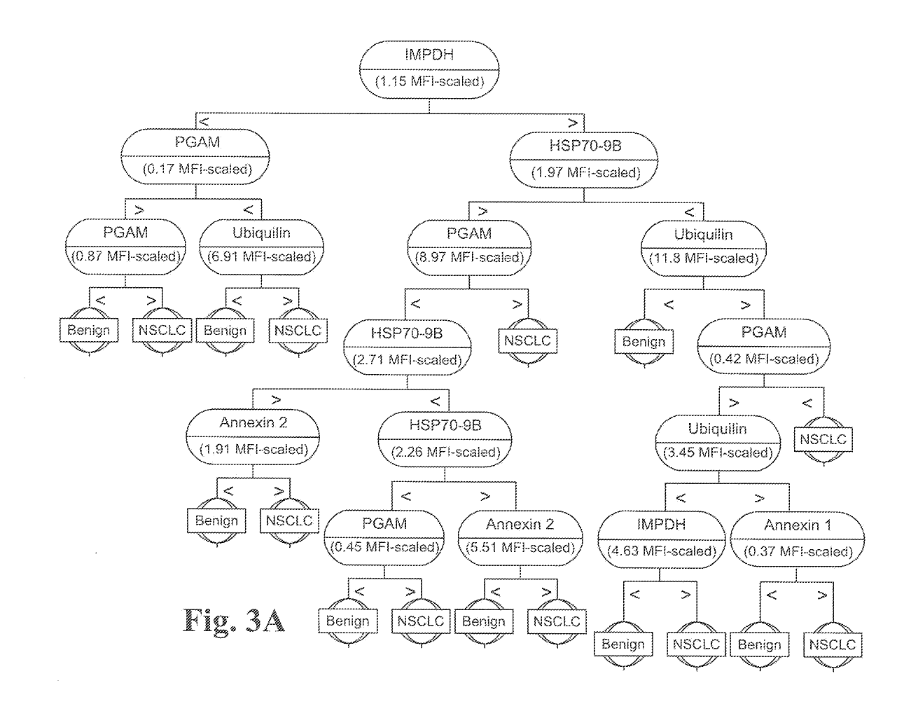

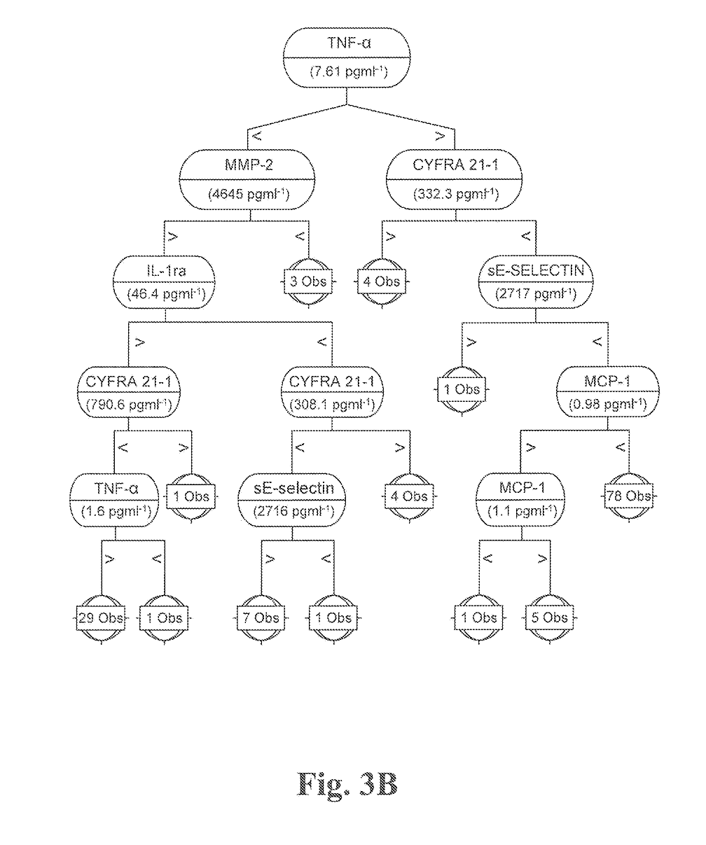

The continued ‘focusing’ of the panel from the 14 individual biomarkers to the six-analyte panel improved the ability to correctly classify patients relative to the pathological NSCLC status. However, after the 4th iteration the AUC and associated sensitivity decreased as the number of biomarkers decreased leading to the selection of this panel as the preferred combination for diagnosing NSCLC. Individual ‘box and whisker’ plots are shown for these six biomarkers (TNF-α, CYFRA 21.1, IL-1ra, MMP-2, MCP-1, and sE-selectin) in FIGS. 2G-2L. Next, a classification tree was defined based on a subpanel of six markers selected from the random forest algorithm within the RPART software package to provide a convenient and useful algorithm for distinguishing NSCLC from benign controls. The classification tree resulting from this process is represented in FIG. 3B. This tree correctly classified 129 of the 135 cases (a correct classification rate of 95%). The ROC curve for this classification tree is shown in FIG. 4. Test performance characteristics for this panel boast a 97.9% AUC translating to 99% sensitivity and 95% specificity. A substantial gain in the ability to screen for NSCLC was identified when using the multianalyte panel over any individual biomarker.

Proteomic Discovery—Cell Culture and Preparation of Cellular Lysates—The human lung adenocarcinoma cell line HCC827 was obtained directly from American Type Culture Collection (ATCC; Manassas, Va.) expressly for the described studies and all studies were performed within six months of the original purchase date. Cell line authentications were performed by ATCC. Cells were grown in RPMI 1640 supplemented with 10% FBS at 37° C. under a humidified atmosphere of 5% CO2. All cells were kept within five passages total for the experiments described. Upon achieving 80% confluency, all cells were harvested and washed twice in PBS, pH 7.4. Cellular lysate were prepared by taking 1×107 cells in 500 μl of 1% NP-40 diluted in TBS supplemented with complete mini-protease inhibitor tablet (Roche Diagnostics; Indianapolis, Ind.). Cells were lysed for 30 min at 4° C., centrifuged for 10 min at 14,000×g, and a protein concentration determined by the BCA method (Pierce; Rockford, Ill.).

Serum autoantigen profiling by 2D Western blot analysis—A total of three gels were run simultaneously for the two-D analysis; two gels were loaded with 100 μg of lysates from the HCC827 cell line and were subjected to Western blot analysis to identify differences in immunoreactivity between lung adenocarcinoma and control patient population, whereas one gel was loaded with 300 μg protein to visualize the protein pattern by gel staining. Whole cell lysate were prepared for this analysis using a ProteoExtract® protein precipitation kit (EMD Chemicals Inc; Gibbstwon, N.J.). Isoelectric focusing (IEF) was conducted using a Protean® IEF cell (BioRad) with the linear gradient program for 22,000-24,000 V-hrs and completed using otherwise standardized protocols recommended by the apparatus manufacturer (BioRad; Hercules, Calif.). After the 2D gels were completed they were either analyzed by 2D Western blot analysis or stained with Gelcode Blue (Pierce Protein Research Products; Rockford, Ill.). For Western blot analysis, proteins from each gel were transferred onto a nitrocellulose membrane at 15V, using standard “tank-transfer” protocols. After blocking with 1% BSA in PBS, membranes were incubated for 1 hour in a 1:500 dilution of pooled sera (10 specimens per group) from either the lung adenocarcinoma or control patient groups (n=10 per group). The probed membranes were then washed with PBS and incubated with HRP-conjugated goat anti-human IgG (Jackson Laboratory; Bar Harbor, Me.) at a 1:100,000 dilution. Immunoblots were developed with the Enhanced ChemiLuminiscence system (ECL; GE Healthcare Bio-Sciences Corp.; Piscataway, N.J.) and documented on X-ray film. All gels and x-ray films were then scanned using a VersaDoc 4000 gel imaging system and compared using PDQuest two-D gel imaging software, version 8.0 (BioRad; Hercules, Calif.). Replicate runs of the gels and immunoblots were performed to ensure reproducibility of observed patterns of immunoreactivity and the targets cored for protein identification.

Sample Preparation for Mass Spectrometry—Differentially immunoreactive signals observed in the Western blots were cored from the Gelcode Blue stained two-D gels for identification via tandem mass spectrometry based on having greater than a ten-fold difference in immunoreactivity. In-gel trypsin digestions were accomplished using methods specified by the Promega Corporation (http://www.promega.com/tbs/tb309/tb309.pdf). After digestion, the resultant peptides were concentrated and desalted using C18 ZipTip (Millipore; Billerica, Mass.) and spotted directly on the MALDI target plate. Recrystallized α-cyano-4-hydroxycinnamic acid (CHCA) (Protea Biosciences; Morgantown, W. Va.) suspended in 50% acetonitrile containing 0.1% trifluoroacetic acid at 5 mg/mL was added to each sample position (1 μL per sample) prior to analysis by tandem mass spectrometry.

Mass Spectrometric Identification of Proteins—Protein identification via mass spectrometry was performed on a Shimadzu AXIMA Performance (MALDI TOF/TOF) mass spectrometer (Shimadzu Biotech; Columbia, Md.) in positive ion mode, optimized to the 700-4000 m/z range. Data was acquired with 2,000 laser shots across each sample. A peptide mass fingerprint (PMF) analysis was performed using the monoisotopic peak list extracted by Mascot Distiller from the raw mass spectrometry files. Peptide matching and protein searches was accomplished against the NCBI and Swiss-Prot databases using the Mascot search engine (Matrix Science; Boston, Mass.) with a mass tolerance set to 100 ppm and 1 missed cleavage with no modifications. In addition, 3-5 unique peptides were selected from the peptides observed in the MS data to perform a MS/MS analysis. Protein identifications were accomplished by importing each peptide sequence tag (PKL) format file (generated by each MS/MS experiments) into the Mascot search engine and used a MS/MS tolerance of ±0.3 Da to search the NCBI and the Swiss-Prot databases.

Criteria for assignment of protein identity consisted of the following parameters: MASCOT ‘MOWSE’ scores greater than 67 (p=0.05), sequence coverage greater than 30%, MS/MS data on a minimum of 3 unique tryptic peptides, and general agreement between predicted and observed mass/isoelectric point (pI) gel coordinate values. Further, 2D Western blots were also performed using commercially-available monoclonal and polyclonal antibodies to confirm that the identified autoantigen correlated with the gel coordinates from which immunoreactivity was originally observed. Antibody sources were as follows: NY-ESO, survivin, recoverin, methylthioadenosine phosphorylase, p53, peroxiredoxin, and triosephosphoisomerarase from Santa Cruz Biotechnology Inc. (Santa Cruz, Calif.); enolase 1 and GAPDH from Abcam Inc. (Cambridge, Mass.); annexin al (R&D systems, Minneapolis, Minn.), calponin 2 from Avia Systems Biology (San Diego, Calif.); hydroxysteroid (17-β) dehydrogenase and phosphoglycerate dehydrogenase from Sigma-Aldrich (St. Louis, Mo.); and the remaining antibodies were from the Abnova Corporation (Taipei City, Taiwan).

Serum test development—Recombinant Proteins: Sources and Production—Recombinant proteins were obtained for each of the candidate autoantibodies identified in the proteomic discovery efforts having value for NSCLC detection (see Table 7) as well as for a second group of autoantibodies with documented value for our purpose, including NY-ESO (13-15), p53 (13, 16-21), peroxiredoxin (22, 23), triosephosphate isomerase (TPI)(23), recoverin (24), 3-oxoacid CoA transferase (23), survivin (also known as BIRC5) (25), c-Myc (13, 26), annexin II (27) and ubiquillin (28). Commercial antibodies (as used above for target confirmation) were obtained for these targets to serve as positive controls during assay performance. A subset of the recombinant proteins (autoantigens) was custom prepared by our collaborators at Abnova Corporation (Taipei City, Taiwan), as defined elsewhere. (29, 30) These include α-enolase, glyoxalase domain containing 4, methylthioadenosine phosphorylase, phosphoglycerate mutase I, IMP-dehydrogenase II, triosephosphate isomerase, recoverin, phosphoglycerate dehydrogenase, erp-29, annexin I, annexin A1 (isoform CRA_b), hydroxysteroid-(17-β)-dehydrogenase 10 isoform 1, fumarate hydratase, heat shock 70 kDa protein 9B (mortalin-2), protein disulfide isomerase-associated 3 precursor, isocitrate dehydrogenase 1 isoform 1, calponin-2, c-Myc, annexin II, 3-oxoacid CoA transferase, and GRP-78 precursor.

TABLE 7
Serum test development -- Recombinant Proteins: Sources and Production

| | | | | Molecular | | Total |
|---|---|---|---|---|---|---|
| | | | | weight | Sequence | number of |
| Spot | | | MOWSE | (kDa) | Coverage | peptides |
| No. | Antigen | Accession No. | Scores | Exp./Obs. | (%) | assigned |
| 1 | Alpha-enolase | GI: 450357 | 228 | 50/47 | 64 | 20 |
| 2 | Glyoxalase domain containing 4 | GI: 16198390 | 112 | 40/33 | 44 | 12 |
| 3 | Glyceraldyhyde-3-phosphate dehydrogenase | GI: 31645 | 211 | 40/36 | 52 | 13 |
| 4 | Methylthioadenosine phosphorylase | GI: 847724 | 148 | 35/31 | 74 | 15 |
| 5 | Phosphoglycerate mutase 1 | GI: 130348 | 193 | 36/28 | 20 | 11, * |
| 6 | IMP-dehydrogenase 2 | GI: 6693016 | 78 | 64/56 | 31 | 19 |
| 7 | Phosphoglycerate dehydrogenase | GI: 23308577 | 103 | 64/57 | 37 | 16 |
| 8 | Endoplasmic reticulum protein 29 (erp 29) | GI: 5803013 | 68 | 36/29 | 45 | 11 |
| 9 | Annexin A1, isoform CRA_b | GI: 19582952 | 113 | 40/40 | 44 | 13 |
| 10 | Annexin 1 | GI: 4502101 | 116 | 40/38 | 46 | 14 |
| 11 | Alpha-enolase | GI: 450357 | 71 | 50/47 | 30 | 10 |
| 12 | Hydrosteroid (17-beta) dehydrogenase 10 | GI: 4758504 | 84 | 27/27 | 37 | 10 |
| 13 | Fumarate hydratase | GI: 19743875 | 83 | 64/54 | 50 | 10 |
| 14 | Isocitrate dehydrogenase 10 isoform 1 | GI: 28178825 | 134 | 50/46 | 40 | 17 |
| 15 | Calponin 2 isoform b | GI: 41327730 | 90 | 40/29 | 37 | 11 |
| 16 | Heat shock 70 kDa protein 9B (mortalin-2) | GI: 21040386 | 94 | 55/50 | 20 | 10, * |
| 17 | Alpha-enolase | GI: 450357 | 104 | 50/47 | 51 | 16 |
| 18 | Protein disulfide isomerase-associated 3 | GI: 21361657 | 232 | 62/55 | 49 | 21 |
| 19 | Enolase 1 variant | GI: 62896593 | 90 | 50/47 | 30 | 11 |
| 20 | GRP78 precursor | GI: 386758 | 183 | 64/72 | 44 | 27 |
| 21 | Alpha-enolase | GI: 450357 | 95 | 50/47 | 43 | 14 |

Custom Luminex immunobead assay development—Microsphere-Antigen Coupling—The “direct-capture” bead based immunoassays were developed using protocols suggested by the Luminex Corporation's suggested protocol. Briefly, between 5 and 10 μg of recombinant protein were conjugated with 5×106 SeroMAPT™ microspheres (Luminex Corporation; Austin Tex.); each with a unique bead region identifier. This was accomplished by activating the microspheres suspended in a solution of sodium phosphate, pH 6.2, containing 5 mg/mL sulfo-NHS (Thermo Scientific; Rockford, Ill.) and 5 mg/mL 1-ethyl-3-(3-dimethylaminopropyl)carbodiimide (EDC) (Thermo Scientific). After a 20 min. incubation in the dark, the microspheres were washed and resuspended in 50 mM two-(N-morpholino)ethanesulfonic acid (MES), pH 5.0, and the appropriate volume of recombinant protein added. The beads were then incubated at ambient temperature in the dark with continuous mixing for 2 hours. Following incubation, the microspheres were then washed twice with PBS containing 0.1% BSA, 0.2% tween-20 and stored in the same buffer at 4° C.

Microsphere Validation—Commercial antibodies (as defined above) were obtained for all candidate autoantigens to serve as a positive control during the direct capture assays. All antigen-coupled microspheres were subjected to individual validation per procedures recommended by the Luminex Corporation. Briefly, serial dilutions of each protein-specific antibody (ranging 4 μg/mL to 0.0625 μg/mL in PBS, 1% BSA) were incubated for 2 hours with 5,000 antigen-conjugated microspheres per well in a 1.2 μm PVDF filter 96-well microtiter plate (Millipore; Billerica, Mass.). Following washing with PBS, 1% BSA, the immobilized autoantibody complex was incubated with 4-6 μg/mL of the biotin-conjugated, anti-human polyclonal antibody (Sigma-Aldrich Co.; St. Louis, Mo.) for 1 hour with constant agitation. Finally, after two washes (as before), the complex was incubated with 4-6 μg/mL R-phycoerythrin conjugated-streptavidin, (Thermo Scientific) for 45 minutes with constant agitation. The resulting complex was again washed and resuspended in PBS, 1% BSA before being read on our Luminex 100 bioanalyzer that uses IS 2.3 software (Luminex Corp.; Austin, Tex.). Performance characteristics were then established for each assay, including range, % CV, sensitivity and specificity, all with in SPSS v15.0.

Following successful individual assay validation, multiplex validation was performed using a modified ‘leave one out’ protocol from Luminex Corporation. The protein-coupled microspheres were grouped into sets containing four to six autoantibody assays per panel selected based on having low protein sequence homology across the group as to avoid cross-reactivity. The median fluorescence intensities (MFI) of each microsphere set (performed as above) were compared to the panel values to confirm that the multiplexed protein-microsphere sets did not have positive interference with one another. Finally, serial dilutions of three stock serum specimens were also used to evaluate the individual microspheres for cross-reactivity.

Serum Analysis using Validated Microsphere Sets—Once multiplexing validation was completed, the resulting five distinct combinations of autoantigen-microsphere panels were used to evaluate the patient serum specimens (n=196) for autoantibody levels. For this assessment, all serum was typically diluted 1:20 using PBS containing 1% BSA and assays otherwise performed as described above. The reported median fluorescence intensity values, which correlate to the concentration of a given autoantibody in the serum, were scaled relative to the MFI values obtained using available monoclonal or polyclonal antibodies as standards. The actual MFI value used for scaling was selected on the basis of being closest to the median MFI value for the entirety of the patient cohort. These are referred to as ‘MFISCALED’ values in the remainder of the manuscript.

Univariate Statistics—Descriptive statistics, receiver operator characteristic (ROC) parameters [including ‘area-under-the curve’ (AUC), specificity and sensitivity] and p-values from Mann-Whitney U test with two comparator groups were obtained for the individual analytes using the SPSS statistical software version 15.0 (SPSS, Chicago, Ill.), as we previously defined.

Multivariate Analysis—An optimized multivariate panel of autoantibody biomarkers was selected from the data resulting from evaluation of the patient cohorts using the Random Forests package as described previously. The optimal panel of biomarkers resulting from the random Forest variable selection process described above was then used by a Classification and Regression Tree (CART) algorithm to model a specific classification system for identifying a patient's cancer status (NSCLC vs. non-NSCLC). This analysis was performed using the RPART package of the R statistical software suite. From the final classification tree, we are able to calculate standard test performance characteristic values (i.e. misclassification error and receiver operator characteristics curve parameters).

Results

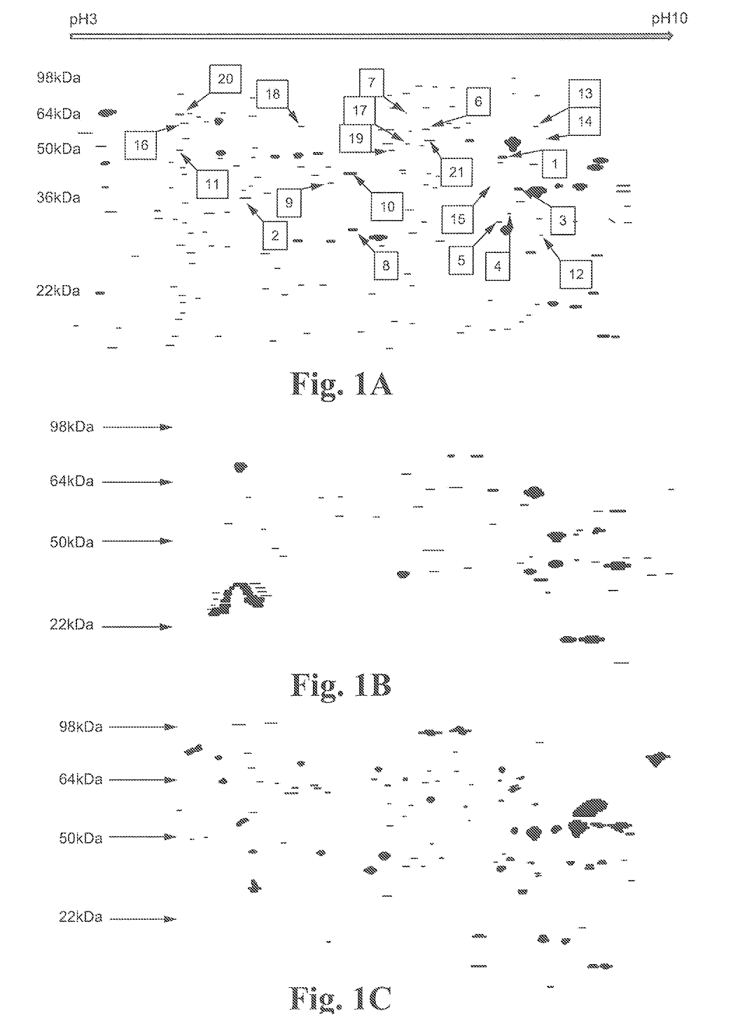

Serum Autoantigen Profiling by 2D Western Blot Analysis—To identify candidate tumor-associated autoantigens differently represented in our two patient groups, we resolved our HCC827 cellular lysates via two-dimensional Western blots, with each immunoblot probed individually with pooled sera from the control and adenocarcinoma patient groups (n=10 per group; see Table 3 for patient characteristics). FIG. 1A shows a representative Coomassie stained 2D gel of the proteins extracted from the lung adenocarcinoma cell line HCC827 with the differences in immunoreactivity to patient sera shown in FIGS. 1B and 1C. A total of 21 spots were selected for identification via tandem mass spectrometry based on possessing a greater than ten-fold difference in immunoreactivity.

Protein Identification via Tandem Mass Spectrometry—Candidate autoantigens recovered from the two-D gels were analyzed on our Shimadzu AXIMA Performance (MALDI-TOF/TOF) mass spectrometer to establish protein identity via standard in-gel digestion methodology. A peptide fingerprint analysis coupled with MS/MS experiments was used to determine the identity of the selected autoantigens and are presented in Table 7. Each target identified by this analysis correlated highly to the predicted gel coordinates (both pI and apparent MW) from which it was originally excised. Intriguingly, the MS/MS data for the spot numbers 12 and 18 identified two proteins for each spot, namely 3-hydroxyacyl-CoA dehydrogenase type 2 and hydrosteroid (17-beta) dehydrogenase 10 isoform 1 for spot 12 and ER-60 protease and protein disulfide isomerase-associated 3 precursor for spot 18. A protein-protein BLAST on the NCBI website demonstrated that each of these pairs shared 100% sequence homology. Also of interest is the observation that isoforms of α-enolase were identified in five different positions (spots 1, 11, 17, 19, and 21) from this analysis, whereas annexin A1 was identified in two positions (spots 9 and 10). These results were confirmed via 2D Western blots using commercially-obtained polyclonal and monoclonal antibodies against each candidate autoantigen. Further, these studies also demonstrated that each gel coordinate contained an immunoreactive protein that corresponded to that shown in FIGS. 3A and 3B (results not shown).

Autoantigen Validation against a Second Patient Cohort—In an effort to come closer to a clinically usable early detection panel for the detection of NSCLC, 15 of the 16 distinct autoantigens identified in our immunoproteomic analysis were translated into Luminex-based immunobead serum assays. The glyoxalase domain containing 4 (GLOD4) was not validated by Luminex assays due to a lack of readily attainable recombinant protein for assay development, though is currently in development. In addition, 10 promising markers (NY-ESO, p53, peroxiredoxin, triosephosphate isomerase, recoverin, 3-oxoacid CoA transferase, survivin, c-Myc, annexin II, and ubiquillin) found in the literature were also translated into Luminex-based immunobead assays by our group. With this, a total of 25 custom immunobead assays were used to evaluate our 117 NSCLC patient serum and 79 control patient serum for circulating autoantibodies specific for NSCLC status. The demographic and clinical characteristics of these cohorts are defined in Table 7.

Of the 15 autoantibodies (identified by this report) that were evaluated, 7 were found to be significantly elevated (AUC greater than 0.60 and a Mann-Whitney U value less than 0.05) in NSCLC patients relative to the control population. These include inosine-5-monophosphate dehydrogenase (IMPDH), fumarate hydratase (FH), α-enolase, endoplasmic reticulum protein 29 (Erp29), annexin 1, hydrosteroid 1743 dehydrogenase, and methylthioadenosine phosphorylase (MTAP). Of those evaluated from the literature, we found annexin II to be the most promising as a biomarker, possessing an AUC of 0.683 and p<0.001. Ubiquilin, c-Myc, NY-ESO, 3-oxoacid CoA transferase and p53 also found to be significant in both the AUC and Mann Whitney two-sided p-value. All other analytes failed to meet statistical significance. Table 8 lists the individual test performance characteristics for the tests against the autoantigens.

TABLE 8
Performance characteristics for the tests against the autoantigens.

| | Mann-Whitney | |
|---|---|---|
| | p-value | ROC |
| Analyte | (two-sided) | AUC |
| Inosine-5-monophosphate dehydrogenase | <0.001 | 0.739 |
| Annexin II | <0.001 | 0.693 |
| Fumarate hydratase | <0.001 | 0.678 |
| Ubiquilin | <0.001 | 0.677 |
| c-MYC | <0.001 | 0.652 |
| α-enolase | 0.0013 | 0.635 |
| NY-ESO | 0.0016 | 0.633 |
| p53 | 0.0023 | 0.628 |
| Endoplasmic reticulum protein 29 (erp 29) | 0.013 | 0.604 |
| 3-oxoacid CoA Transferase | 0.014 | 0.604 |
| Methylthioadenosine phosphorylase | 0.018 | 0.6 |
| Annexin I | 0.019 | 0.599 |
| Hydroxysteroid (17-β) dehydrogenase 10 | 0.026 | 0.594 |
| Heat shock protein 70 kDA protein 9B | 0.088 | 0.572 |
| (mortalin-2) | | |
| phosphoglycerate mutase | 0.09 | 0.571 |
| Recoverin | 0.177 | 0.557 |
| Heat shock protein 5 (GRP78 precursor) | 0.186 | 0.556 |
| Peroxiredoxin | 0.202 | 0.554 |
| Calponin 2 | 0.204 | 0.554 |
| Phosphoglycerate dehydrogenase | 0.336 | 0.541 |
| Protein disulfide isomerase 3 | 0.33 | 0.541 |
| Glyceraldehyde-3-phosphate dehydrogenase | 0.366 | 0.538 |
| Triosephosphate isomerarase | 0.361 | 0.538 |
| Survivin | 0.625 | 0.521 |
| Isocitrate dehydrogenase | 0.636 | 0.52 |

Validating the performance characteristics of this six-analyte panel against a second patient cohort, 75 of 88 patients were successfully classified. An examination of the individual groups was then performed as a means to confirm the relevance of the associations of the individual biomarkers with promise for the panel to screen for NSCLC. When looking solely at the cohort composed of COPD and asthma patients, only a single patient was misclassified (false positive) of the 40 tested. In the NSCLC cohort, five patients were misclassified of the 33 patients, resulting in an 85% classification rate. Misclassifications were not limited to Stage 1A patients, indicating errors were not due to test sensitivity. And finally, only eight of the 15 patients with resected, non-neoplastic disease were correctly classified. This sub-group may require further development in order to improve the range of patients that can be accurately classified by this methodology.

The human lung adenocarcinoma cell line HCC827 was obtained directly from American Type Culture Collection (ATCC; Manassas, Va.) expressly for the described studies and all studies were performed within six months of the original purchase date. Cell line authentications were performed by ATCC. Cells were grown in RPMI 1640 supplemented with 10% FBS at 37° C. under a humidified atmosphere of 5% CO2. All cells were kept within five passages total for the experiments described. Upon achieving 80% confluency, all cells were harvested and washed twice in PBS, pH 7.4. Cellular lysate were prepared by taking 1×107 cells in 500 μl of 1% NP-40 diluted in TBS supplemented with complete mini-protease inhibitor tablet (Roche Diagnostics; Indianapolis, Ind.). Cells were lysed for 30 min. at 4° C., centrifuged for 10 min at 14,000×g, and a protein concentration determined by the BCA method (Pierce; Rockford, Ill.).

A total of three gels were run simultaneously for the two-D analysis; two gels were loaded with 100 μg of lysates from the HCC827 cell line and were subjected to Western blot analysis to identify differences in immunoreactivity between lung adenocarcinoma and control patient population, whereas one gel was loaded with 300 μg protein to visualize the protein pattern by gel staining. Whole cell lysates were prepared for this analysis using a ProteoExtract® protein precipitation kit (EMD Chemicals Inc; Gibbstwon, N.J.) following its instructions for use. Isoelectric focusing (IEF) was conducted using a Protean® IEF cell (BioRad) with the linear gradient program for 22,000-24,000 V-hrs and completed using otherwise standardized protocols recommended by the apparatus manufacturer (BioRad; Hercules, Calif.). After the 2D gels were completed they were either analyzed by 2D Western blot analysis or stained with Gelcode Blue (Pierce Protein Research Products; Rockford, Ill.). For Western blot analysis, proteins from each gel were transferred onto a nitrocellulose membrane at 15V, using standard “tank-transfer” protocols. After blocking with 1% BSA in PBS, membranes were incubated for 1 hour in a 1:500 dilution of pooled sera (10 specimens per group) from either the lung adenocarcinoma or control patient groups (n=10 per group). The probed membranes were then washed with PBS and incubated with HRP-conjugated goat anti-human IgG (Jackson Laboratory; Bar Harbor, Me.) at a 1:100,000 dilution. Immunoblots were developed with the Enhanced ChemiLuminiscence system (ECL; GE Healthcare Bio-Sciences Corp.; Piscataway, N.J.) and documented on X-ray film. All gels and x-ray films were then scanned using a VersaDoc 4000 gel imaging system and compared using PDQuest two-D gel imaging software, version 8.0 (BioRad; Hercules, Calif.). Replicate runs of the gels and immunoblots were performed to ensure reproducibility of observed patterns of immunoreactivity and the targets cored for protein identification.

Differentially immunoreactive signals observed in the Western blots were cored from the Gelcode Blue stained two-D gels for identification via tandem mass spectrometry based on having greater than a ten-fold difference in immunoreactivity. In-gel trypsin digestions were accomplished using methods specified by the Promega Corporation. After digestion, the resultant peptides were concentrated and desalted using C18 ZipTip (Millipore; Billerica, Mass.) and spotted directly on the MALDI target plate. Recrystallized α-cyano-4-hydroxycinnamic acid (CHCA) (Protea Biosciences; Morgantown, W. Va.) suspended in 50% acetonitrile containing 0.1% trifluoroacetic acid at 5 mg/mL was added to each sample position (1 μL per sample) prior to analysis by tandem mass spectrometry.

Protein identification via mass spectrometry was performed on a Shimadzu AXIMA Performance (MALDI TOF/TOF) mass spectrometer (Shimadzu Biotech; Columbia, Md.) in positive ion mode, optimized to the 700-4000 m/z range. Data was acquired with 2,000 laser shots across each sample. A peptide mass fingerprint (PMF) analysis was performed using the monoisotopic peak list extracted by Mascot Distiller from the raw mass spectrometry files. Peptide matching and protein searches was accomplished against the NCBI and Swiss-Prot databases using the Mascot search engine (Matrix Science; Boston, Mass.) with a mass tolerance set to 100 ppm and 1 missed cleavage with no modifications. In addition, 3-5 unique peptides were selected from the peptides observed in the MS data to perform a MS/MS analysis. Protein identifications were accomplished by importing each peptide sequence tag (PKL) format file (generated by each MS/MS experiments) into the Mascot search engine and used a MS/MS tolerance of ±0.3 Da to search the NCBI and the Swiss-Prot databases.

Criteria for assignment of protein identity consisted of the following parameters: MASCOT ‘MOWSE’ scores greater than 67 (p=0.05), sequence coverage greater than 30%, MS/MS data on a minimum of 3 unique tryptic peptides, and general agreement between predicted and observed mass/isoelectric point (pI) gel coordinate values. Further, 2D Western blots were also performed using commercially-available monoclonal and polyclonal antibodies to confirm that the identified autoantigen correlated with the gel coordinates from which immunoreactivity was originally observed. Antibody sources were as follows: NY-ESO, survivin, recoverin, methylthioadenosine phosphorylase, p53, peroxiredoxin, and triosephosphoisomerarase from Santa Cruz Biotechnology Inc. (Santa Cruz, Calif.); enolase 1 and GAPDH from Abcam Inc. (Cambridge, Mass.); annexin al (R&D systems, Minneapolis, Minn.), calponin 2 from Avia Systems Biology (San Diego, Calif.); hydroxysteroid (17-β) dehydrogenase and phosphoglycerate dehydrogenase from Sigma-Aldrich (St. Louis, Mo.); and the remaining antibodies were from the Abnova Corporation (Taipei City, Taiwan).

Recombinant proteins were obtained for each of the candidate autoantibodies identified in the proteomic discovery efforts having value for NSCLC detection (see Table 7) as well as for a second group of autoantibodies with documented value for our purpose, including NY-ESO, p53, peroxiredoxin, triosephosphate isomerase (TPI), recoverin, 3-oxoacid CoA transferase, survivin (also known as BIRC5), c-Myc, annexin II and ubiquillin. Commercial antibodies (as used above for target confirmation) were obtained for these targets to serve as positive controls during assay performance. A subset of the recombinant proteins (autoantigens) was custom prepared by Abnova Corporation (Taipei City, Taiwan). These include α-enolase, glyoxalase domain containing 4, methylthioadenosine phosphorylase, phosphoglycerate mutase I, IMP-dehydrogenase II, triosephosphate isomerase, recoverin, phosphoglycerate dehydrogenase, erp-29, annexin I, annexin A1 (isoform CRA_b), hydroxysteroid-(17-β)-dehydrogenase 10 isoform 1, fumarate hydratase, heat shock 70 kDa protein 9B (mortalin-2), protein disulfide isomerase-associated 3 precursor, isocitrate dehydrogenase 1 isoform 1, calponin-2, c-Myc, annexin II, 3-oxoacid CoA transferase, and GRP-78 precursor.

The “direct-capture” bead based immunoassays were developed using protocols suggested by the Luminex Corporation's suggested protocol. Between 5 and 10 μg of recombinant protein were conjugated with 5×106 SeroMAP™ microspheres (Luminex Corporation; Austin Tex.); each with a unique bead region identifier. This was accomplished by activating the microspheres suspended in a solution of sodium phosphate, pH 6.2, containing 5 mg/mL sulfo-NHS (Thermo Scientific; Rockford, Ill.) and 5 mg/mL 1-ethyl-3-(3-dimethylaminopropyl)carbodiimide (EDC) (Thermo Scientific). After a 20 min. incubation in the dark, the microspheres were washed and resuspended in 50 mM two-(N-morpholino) ethanesulfonic acid (MES), pH 5.0, and the appropriate volume of recombinant protein added. The beads were then incubated at ambient temperature in the dark with continuous mixing for 2 hours. Following incubation, the microspheres were then washed twice with PBS containing 0.1% BSA, 0.2% tween-20 and stored in the same buffer at 4° C.

Commercial antibodies were obtained for all candidate autoantigens to serve as a positive control during the direct capture assays. All antigen-coupled microspheres were subjected to individual validation per procedures recommended by the Luminex Corporation. Serial dilutions of each protein-specific antibody (ranging 4 μg/mL to 0.0625 μg/mL in PBS, 1% BSA) were incubated for 2 hours with 5,000 antigen-conjugated microspheres per well in a 1.2 μm PVDF filter 96-well microtiter plate (Millipore; Billerica, Mass.). Following washing with PBS, 1% BSA, the immobilized autoantibody complex was incubated with 4-6 μg/mL of the biotin-conjugated, anti-human polyclonal antibody (Sigma-Aldrich Co.; St. Louis, Mo.) for 1 hour with constant agitation. Finally, after two washes (as before), the complex was incubated with 4-6 μg/mL R-phycoerythrin conjugated-streptavidin, (Thermo Scientific) for 45 minutes with constant agitation. The resulting complex was again washed and resuspended in PBS, 1% BSA before being read on our Luminex 100 bioanalyzer that uses IS 2.3 software (Luminex Corp.; Austin, Tex.). Performance characteristics were then established for each assay, including range, % CV, sensitivity and specificity, all with in SPSS v15.0.

Following successful individual assay validation, multiplex validation was performed using a modified ‘leave one out’ protocol from Luminex Corporation. The protein-coupled microspheres were grouped into sets containing four to six autoantibody assays per panel selected based on having low protein sequence homology across the group as to avoid cross-reactivity. The median fluorescence intensities (MFI) of each microsphere set (performed as above) were compared to the panel values to confirm that the multiplexed protein-microsphere sets did not have positive interference with one another. Finally, serial dilutions of 3 stock serum specimens were also used to evaluate the individual microspheres for cross-reactivity.

Once multiplexing validation was completed, the resulting five distinct combinations of autoantigen-microsphere panels were used to evaluate the patient serum specimens (n=196) for autoantibody levels. For this assessment, all serum was typically diluted 1:20 using PBS containing 1% BSA and assays otherwise performed as described above. The reported median fluorescence intensity values, which correlate to the concentration of a given autoantibody in the serum, were scaled relative to the MFI values obtained using available monoclonal or polyclonal antibodies as standards. The actual MFI value used for scaling was selected on the basis of being closest to the median MFI value for the entirety of the patient cohort. These are referred to as ‘MFISCALED’ values in the remainder of the manuscript.

To identify candidate tumor-associated autoantigens differently represented in the two patient groups, HCC827 cellular lysates were resolved via two-dimensional Western blots, with each immunoblot probed individually with pooled sera from the control and adenocarcinoma patient groups (n=10 per group; FIG. 1A shows a representative Coomassie stained 2D gel of the proteins extracted from the lung adenocarcinoma cell line HCC827 with the differences in immunoreactivity to patient sera shown in FIGS. 1B and 1C. A total of 21 spots were selected for identification via tandem mass spectrometry based on possessing a greater than ten-fold difference in immunoreactivity.

Candidate autoantigens recovered from the two-D gels were analyzed on a Shimadzu AXIMA Performance (MALDI-TOF/TOF) mass spectrometer to establish protein identity via standard in-gel digestion methodology. A peptide fingerprint analysis coupled with MS/MS experiments was used to determine the identity of the selected autoantigens and are presented in Table 5. Each target identified by this analysis correlated highly to the predicted gel coordinates (both pI and apparent MW) from which it was originally excised. The MS/MS data for the spot numbers 12 and 18 identified two proteins for each spot, namely 3-hydroxyacyl-CoA dehydrogenase type 2 and hydrosteroid (17-beta) dehydrogenase 10 isoform 1 for spot 12 and ER-60 protease and protein disulfide isomerase-associated 3 precursor for spot 18. A protein-protein BLAST on the NCBI website demonstrated that each of these pairs shared 100% sequence homology. Isofroms of α-enolase were identified in five different positions (spots 1, 11, 17, 19, and 21) from this analysis, whereas annexin A1 was identified in two positions (spots 9 and 10). These results were confirmed via 2D Western blots using commercially-obtained polyclonal and monoclonal antibodies against each candidate autoantigen. Further, these studies demonstrated that each gel coordinate contained an immunoreactive protein that corresponded to that shown in FIG. 1A.

Fifteen of the sixteen distinct autoantigens identified in the immunoproteomic analysis were translated into Luminex-based immunobead serum assays. Ten additional markers (NY-ESO, p53, peroxiredoxin, triosephosphate isomerase, recoverin, 3-oxoacid CoA transferase, survivin, c-Myc, annexin II, and ubiquillin) were translated into Luminex-based immunobead assays. With this, a total of 25 custom immunobead assays were used to evaluate 117 NSCLC patient serum and 79 control patient serum for circulating autoantibodies specific for NSCLC status.

Of the 15 autoantibodies that were evaluated, seven were found to be significantly elevated (AUC greater than 0.60 and a Mann-Whitney U value less than 0.05) in NSCLC patients relative to the control population. These include inosine-5-monophosphate dehydrogenase (IMPDH), fumarate hydratase (FH), α-enolase, endoplasmic reticulum protein 29 (Erp29), annexin 1, hydrosteroid 17-P dehydrogenase, and methylthioadenosine phosphorylase (MTAP). Of the ten additional markers, annexin II was the best biomarker, possessing an AUC of 0.683 and p<0.001. Ubiquilin, c-Myc, NY-ESO, 3-oxoacid CoA transferase and p53 also found to be significant in both the AUC and Mann Whitney two-sided p-value. All other analytes failed to meet statistical significance. Table 3 lists the individual test performance characteristics for the tests against the autoantigens.

With the Random Forest multivariate analysis, a panel of six analytes [IMPDH, phosphoglycerate mutase (PGAM), ubiquillin, annexin I, annexin II, and heat shock protein 70-9B (HSP70-9B)] was determined to be the optimal combination of analytes for distinguishing NSCLC patients from the cancer-free controls (with an observed ROC parameter of AUC of 93.4%). Box and whisker plots for these six analytes are shown in FIGS. 2A-2F with the cohorts partitioned. No correlation with age, gender, or smoking status was observed in any of these six analytes that were stronger than those observed according to disease status. A Classification and Regression Tree (CART) analysis was then fashioned from these analytes and used to define a specific algorithm for classifying patients according to NSCLC disease status (see FIG. 3A). This algorithm provided “excellent” ROC parameters for the overall study, including an ‘area-under-the-curve’ of 0.964, a sensitivity of 94.8%, and specificity of 91.1%. The overall misclassification rate was 7% against the entire patient population (n=196). The general agreement of the results yielded by the two multivariate algorithms (i.e. random forest and CART) serves as a confirmation for the specific six-analytes selected by each method.

With the patient cohorts evaluated, a serum test consisting of TNF-α, CYFRA 21.1, IL-1ra, MMP-2, MCP-1, sE-selectin was identified. The cytokeratin 19 fragment, CYFRA 21.1, is perhaps the most extensively characterized biomarker with diagnostic value for NSCLC. Numerous studies have been focused on evaluating its potential for early detection of NSCLC as well as its potential prognostic and predictive value. Each of the remaining analytes has also all been previously implicated individually as having either diagnostic value or a role in inflammation, either in NSCLC or other carcinomas. More specifically, TNF-α, and IL-1ra are both considered to be acute phase reactants and as such, they are involved with modulating the immune response and show increased expression in an inflammatory state. Cancer cells are immunogenic and therefore, lead to the increased expression of proinflammatory agents as well as associated secondary biomarkers. There is an association between chronic inflammation and tumorigenesis, largely due to increases in cell turnover, which can increase serum biomarkers. Similarly, sE-selectin is a cell adhesion molecule, frequently modulated by inflammation. MMP-2 is involved in the degradation of proteins in the extracellular matrix during tissue remodeling for epithelial reorganization.

In terms of performance against the sub-populations within the validation cohorts, the multi-variate panel was able to correctly classify most patients with NSCLC as having NSCLC (15% false-negative rate), as well as patients within the Abbott cohort (2.5% false-positive rate) as not having NSCLC. The sub-population that was the most difficult to classify correctly was the patients with resected non-neoplastic lung disease. Of the patients from this group that were misclassified (47% rate of false-positives); all had an inflammatory condition (i.e. pneumonia, pulmonary abscess, hepatitis C) that may have (at least in part) mimicked the biomarker profile that classifies patients as having NSCLC. This provides a further embodiment, wherein the assay is used on patients with resected non-neoplastic lung disease in combination with available techniques to detect an inflammatory condition to further select patients from this pool with high correlations for diagnosis, outcome and therapeutic selection.

Previous to the report of the panel presented here, the combination of CEA, CA125, CA 19-9, CYFRA 21-1, and NSE was the most efficacious serum test for diagnosing NSCLC, with reported test performance characteristics of a 93.8% sensitivity and 71.5% specificity. Although this panel offers excellent sensitivity, it has poor specificity, making it incapable of serving as a means to complement spiral CT-based screening protocols and inadequate to serve as a “stand-alone” diagnostic method.

Based on the results presented here, an NSCLC detection algorithm based on six serum biomarkers is a low-cost and minimally invasive screening test for patients at high risk for NSCLC. To further increase the sensitivity and specificity of this panel, the addition of autoantibodies to the present panel is contemplated. The addition of biomarkers of this type offers the test specificity necessary to discern patients with inflammatory nodules requiring resection from the cases of NSCLC.

For differentiation of NSCLC versus Non-NSCLC, a large number of potential tumor autoantibodies for NSCLC was tested and validated for utility. Three of these autoantibodies [methylthioadenosine phosphorylase (MTAP), fumarate hydratase (FH), and the endoplasmic reticulum protein 29 (Erp29)] represent new autoantigen targets for distinguishing NSCLC from control populations. Of the analytes previously seen in the literature, inosine-5-monophosphate dehydrogenase (IMPDH), annexin II, ubiquilin, c-Myc, and α-enolase showed the most promise (AUC>0.63) for application in the early diagnosis of NSCLC.

The six-analyte blood test that resulted from the present study (consisting of IMPDH, phosphoglycerate mutase, ubiquillin, annexin I, annexin II, and HSP70-9B) possesses excellent test performance characteristics when tested against the 196 patient-cohort that was composed of four clinically distinct groups, with only 13 patients misclassified overall. Within the classification errors we encountered, we observed a 4% false-positive rate in the non-NSCLC cohorts, which includes 2 patients from the Abbott cohort (6% of group; 1-asthma, 1-COPD), 1 patient from the “osteoarthritis” cohort (3% of group), and 4 patients from the cohort that had resected “non-neoplastic” nodules (25% of group; two-pneumonia, 1 pneumonitis, and 1 granulomas). No clinical features helped explain the high rate of misclassification within any of these groups. These findings support the idea that at this time the algorithm is well-suited to help indicate which symptomatic patients should have further diagnostic evaluations performed. The high rate of misclassifications within the resected non-neoplastic group may be partially accounted for by the fact that specific inflammatory conditions, such as interstitial lung disease, COPD, and asthma, has been reported to induce the production of autoantibodies that may be common to the autoantibodies found in patients with NSCLC. However, whether these autoantibodies are produced before or during carcinogenesis is not known at this time and it may follow that a subset of these targets may provide a means to early disease detection. This subject will be further pursued in future studies. It should also be noted that the number of patients in this resected non-neoplastic patient cohort was relatively low (16 total) making it difficult to extrapolate the significance of this general finding. Also, given this entire cohort was incorrectly resected for suspected NSCLC, this group may be the most challenging to discern by any current diagnostic method. Even so, we successfully classified 75% of these patients, demonstrating there is value to this approach that complements current clinical and radiographic diagnostic criteria. Perhaps of more concern is the observed 5% (3.1% overall) false negative rate (six of 117) observed in the NSCLC cohort. Although these misclassifications were not clustered around a single sub-group or clinical parameter, there was a higher incidence (four total) of patients with poorly-differentiated tumors within this category. The relevance of this observation is still under investigation. Also of interest is that these misclassifications were not limited to stage 1A patients (2-T1N0M0, 4-T2N0M0), possibly indicating errors were not due to test sensitivity. Along these lines was the finding that the histology of these misclassifications was equally distributed between squamous cell carcinoma (SCC) and adenocarcinoma despite the fact that our test was originally directed exclusively against adenocarcinoma specimens. This shows the targets identified were general to the production of autoantibodies for NSCLC. Interestingly, all SCC patients possessed tumors with poor differentiation upon pathological examination. This is particularly important since the numbers of patients with non-adenocarcinoma histology is substantial (˜40%) in the general population of NSCLC patients.

Multianalyte autoantibody panels have previously been reported for use in NSCLC, but the panel described herein has test performance characteristics for detection of NSCLC superior to any serum test reported. In addition, several of the multi-analyte panels proposed have very small patient populations relative to that reported here.

A suitable diagnostic for the targeted autoantibodies, nucleic acids or polypeptides can be presented in any of a variety of known assay formats. For example, in autoantibody assays, an analyte or epitope can be affixed to a solid phase, for example, using known chemistries. Alternatively, the analytes or epitopes can be conjugated to another molecule, typically larger than the epitope to form a synthetic conjugate molecule or can be made as a composite molecule using recombinant methods, as known in the art. Many polypeptides naturally bind to plastic surfaces, such as polyethylene surfaces, which can be found in tissue culture devices, such as multiwell plates. Such plastic surfaces can be treated as is known in the art to enhance binding of biologically compatible molecules thereto. The polypeptides form a capture element, a liquid suspected of carrying an autoantibody that specifically binds that analyte or epitope is exposed to the capture element, antibody becomes affixed and immobilized to the capture element, and then following a wash, bound antibody is detected using a suitable detectably labeled reporter molecule, for example, using an anti-human antibody labeled with a colloidal metal, a fluorochome, or other appropriate reported as are known in the art.

Alternatively, as particular phage express, an epitope specifically bound by autoantibodies found in patients with NSCLC, the capture element of an assay can be the individual phage, such as obtained from a cell lysate, each at a capture site on a solid phase. A reactively inert carrier, such as a protein (e.g. albumin, keyhole limpet hemocyanin, etc.), or a synthetic carrier (e.g. synthetic polymer, etc.), to which the expressed analyte or epitope is attached, or any other means to present an analyte or epitope of interest on the solid phase for an immunoassay, can be used.

An acceptable assay format may take the configuration wherein a capture element (Protein A, Protein G, etc.) affixed to a solid phase binds to the non-antigen-binding portions of immunoglobulin. Patient sputum, tissue or plasma is exposed to the capture reagent and then presence of the NSCLC specific autoantibody is detected using, for example, labeled marker in a direct or competition format, as known in the art. The capture element can be an antibody which binds the phage displaying the epitope to provide another means to produce a specific capture reagent, as discussed above.

As known in the immunoassay art, the capture element is a determinant to which an antibody binds. As taught herein, the determinant is a biological molecule, or portion thereof, such as a polypeptide, polynucleotide, lipid, polysaccharide, and so on, and combinations thereof, such as glycoprotein or a lipoprotein, selected from the biomarkers described herein, the presence of which correlates with presence of an autoantibody found in NSCLC patients. The determinant can be naturally occurring, recombinant or synthetically manufactured and purified.

The solid phase of an immunoassay can be any of those known in the art, and in forms as known in the art. Thus, the solid phase can be a plastic, such as polystyrene or polypropylene, a glass, a silica-based structure, such as a silicon chip, a membrane, such as nylon, a paper, etc. The solid phase can be presented in a number of different and known formats, such as in paper format, a bead, as part of a dipstick or lateral flow device, which generally employs membranes, a microtiter plate, a slide, a chip and so on. The solid phase can present as a rigid planar surface, as found in a glass slide or on a chip. Some automated detector devices have dedicated disposables associated with a means for reading the detectable signal, for example, a spectrophotometer, liquid scintillation counter, colorimeter, fluorometer and the like for detecting and reading a photon-based signal.

Other immune reagents for detecting the bound antibody are known in the art. For example, an anti-human Ig antibody would be suitable for forming a sandwich comprising the capture determinant, the autoantibody and the anti-human Ig antibody. The anti-human Ig antibody, the detector element, can be directly labeled with a reporter molecule, such as an enzyme, a colloidal metal, radionuclide, a dye and so on, or can itself be bound by a secondary molecule that serves the reporter function. Any means for detecting bound antibody can be used in the exemplary assay described herein, and such any means can contain any means for a reporting function to yield a signal discernable by the operator. The labeling of molecules to form a reporter is known in the art.

In the context of a device that enables the simultaneous analysis of a multitude of samples, a number of control elements, both positive and negative controls can be included on the assay device to enable controlling for assay performance, reagent performance, specificity and sensitivity. Often, as mentioned, much, if not all of the steps in making the device of interest and many of the assay steps can be conducted by a mechanical or automated means. The data from these devices can be digitized by a scanning means, the digital information is communicated to a data storage means and the data also communicated to a data processing means, where the sort of statistical analysis as is known in the art, can be used on the data to produce a measurement outcome or result, which then can be compared to a reference standard or internally compared to present with an assay result by a data presentation means, such as a screen or read out of information, to provide diagnostic information.

For devices which analyze a smaller number of samples or where sufficient population data are available, a derived metric for what constitutes a positive result and a negative result, with appropriate error measurements, can be provided. In those cases, a single positive control and a single negative control may be all that is needed for internal validation, as known in the art. The assay device can be configured to yield a more qualitative result, either included or not in a NSCLC cluster.

Other high throughput and/or automated immunoassay formats can be used as known and available in the art. Thus, for example, a bead-based assay, grounded, for example, on colorimetric, fluorescent or luminescent signals, can be used, such as the Luminex described herein, technology relying on dye-filled microspheres and the Cytometric Bead Array system. In either case, the epitopes of interest are affixed to a bead.

In another example of an assay to detect the biomarker panels described herein, the disclosure identifies the global changes in gene expression associated with a lung cancer such as NSCLC by examining gene expression in tissue from a patient using techniques that are known in the art. Gene expression profiles of the biomarkers described herein can serve as diagnostic markers that can be used to monitor lung cancer disease states, disease progression and drug efficacy. The disclosed embodiments include methods of diagnosing the presence or absence of lung cancer in a patient comprising the step of detecting the level of expression in a tissue sample of two or more genes from Table 6 wherein differential expression of the genes in Table 6 is indicative of a lung cancer such as NSCLC. In some embodiments, one or more genes may be selected from a group consisting of the genes listed in Table 6.

The disclosed embodiments also include methods of detecting the progression of lung cancer and/or differentiating cancerous disease from chronic inflammation. For instance, disclosed methods include detecting the progression of lung cancer in a patient comprising the step of detecting the level of expression in a tissue sample of one or more genes from Table 6 wherein differential expression of the genes in Table 6 is indicative of lung cancer progression.

In some aspects, a method of monitoring the treatment of a patient with a lung cancer such as NSCLC is disclosed, comprising administering a pharmaceutical composition to the patient, preparing a gene expression profile from a cell or tissue sample from the patient and comparing the patient gene expression profile to a gene expression from a cell population comprising normal plasma, sputum or normal lung cells or to a gene expression profile from a cell population comprising serum, sputum or tissue from a patient with lung cancer. In some embodiments, the gene profile will include the expression level of one or more genes in Table 6. In other embodiments, one or more genes may be selected from a group consisting of the genes listed in Table 6.

In another aspect, a method of treating a patient with a lung cancer such as NSCLC is disclosed, comprising administering to the patient a pharmaceutical composition, wherein the composition alters the expression of at least one gene in Table 6, preparing a gene expression profile from a cell or tissue sample from the patient comprising tumor cells and comparing the patient expression profile to a gene expression profile from an untreated cell population comprising lung cancer tissue, urine, serum or sputum.

In another aspect, a test for patient prognostication for recurrence of disease is also disclosed using the methodology disclosed herein. Disease stage provides critical prognostic information for NSCLC patients and guides therapeutic decisions. Roughly 20-30% of NSCLC patients present with localized disease and are eligible for a complete anatomic resection with systematic lymph node dissection as the best possible means to a cure. As the standard of care, patients with locoregional metastases will receive systemic adjuvant chemotherapy as a means to improve outcome. Patients with no apparent metastatic lesions, on the other hand, have a more favorable prognosis than the previous group and receive only disease surveillance after a definitive resection if their tumors were less than 4 cm. Despite these favorable odds, patients with Stage I disease are at high risk for relapse and approximately 30-40% of these patients will die from recurrent disease within 5-years of tumor resection. Recurrent disease is primarily attributed to the presence of occult “micrometastatic” lesions at the time of surgery that were undetected by standard clinical and pathological staging protocols. Although populations of unselected Stage I patients given chemotherapy were shown to trend towards inferior outcomes in clinical trials, the group of early stage patients with occult metastases may receive a significant outcome benefit (similar to the higher stage groups) if effective methods were available for definitive treatment selection.

Currently, there are no validated methods capable of identifying the high risk subset of stage I patients. A growing body of evidence suggests that metastatic progression in epithelial cancers may be driven by a phenotypic shift that globally impacts regulatory pathways and results in aggressive tumor-cell behavior (e.g. highly mobile, anchorage independent and invasive). Our study hypothesis contends that metastasis-specific changes in cellular phenotype also results in differences in tumor-shed protein biomarkers found in the serum. These biomarkers may have great value for detecting occult metastatic disease and predicting patient outcome. Our approach to evaluate this idea will be to build a model of the serum “secretome” for metastatic NSCLC based on a comparative study of metastasis-associated differences in the serum proteome with collated expression array data from almost 500 patients involved in clinical trials evaluating patient outcome in resectable, early-stage NSCLC. A calculated sampling of biomarkers representative of the range of biological pathways most highly-modulated with tumor metastasis will then be used to develop a serum test to select high risk stage I patients who are candidates for systemic adjuvant studies.

The recent observation that CT screening reduces lung cancer mortality will likely result in an increasing number of individuals being diagnosed with stage I lung cancer. Reliable diagnostic methods that identify stage I patients who have a high risk of disease recurrence and are candidates for trials testing systemic adjuvant therapy could result in further reduction in lung cancer mortality. The objectives of this study fill this clinical need by developing a validated serum test capable of stratifying NSCLC patients by outcome and serve as a means to select patients for systemic adjuvant treatment.

Disease stage upon presentation provides the most important prognostic information for these patients and helps guide therapeutic strategy selection. Typically 20-30% of NSCLC patients present with localized disease and are eligible for a complete anatomic resection (R0 resection) with systematic lymph node dissection as the best possible means to a cure. Based on randomized controlled trials and the Lung Adjuvant Cisplatin Evaluation (LACE) meta-analysis, post-operative systemic chemotherapy is recommended for patients with lymph node metastases to improve outcomes when the primary tumors are greater than 4 centimeters. Patients no apparent metastatic lesions, on the other hand, have a more favorable prognosis than the previous group and receive only disease surveillance after a definitive resection if their tumors were less than 4 cm. Despite these favorable odds, patients with Stage I disease are at high risk for relapse and approximately 30-40% of these patients will die from recurrent disease within 5-years of tumor resection. Recurrent disease is primarily attributed to the presence of occult “micrometastatic” lesions at the time of surgery that were undetected by standard clinical and pathological staging protocols. Although populations of unselected Stage I patients given chemotherapy were shown to trend towards inferior outcomes in clinical trials, the group of early stage patients with occult metastases may receive a significant outcome benefit (similar to the higher stage groups) if effective methods were available for definitive treatment selection.

A growing body of evidence also suggests that metastatic progression in epithelial cancers may be driven by a phenotypic transition towards one with features of mesenchymal stem cells. Molecular characteristics associated with this Epithelial-to-Mesenchymal Transition (EMT) include globally impacts regulatory pathways and results in aggressive tumor-cell behavior (e.g. highly mobile, anchorage independent and invasive). Without being bound by theory, it is contended that metastasis-specific changes in cellular phenotype also results in differences in tumor-shed protein biomarkers found in the serum. These biomarkers may have great value for detecting occult metastatic disease and predicting patient outcome. The approach to evaluate this idea is be to build a model of the serum “secretome” for metastatic NSCLC based on a comparative study of metastasis-associated differences in the serum proteome with collated expression array data from almost 500 patients involved in clinical trials evaluating patient outcome in resectable, early-stage NSCLC. A calculated sampling of biomarkers representative of the range of biological pathways most highly-modulated with tumor metastasis will then be used to develop a serum test to select high risk stage I patients who are candidates for systemic adjuvant studies. We propose three specific aims to achieve these goals:

Aim 1: Identifying biomarkers that have value for classifying Stage I NSCLC patients based on risk for disease recurrence. This aim will be accomplished by intersecting data from the serum proteome (n=100) with data from approximately 500 reposited expression microarray profiles recently acquired from studies evaluating survival prediction for patients with resectable NSCLC.

Aim 2: Measuring (absolute quantitation) circulating levels of no less than 25 candidate biomarkers identified above and evaluate these candidate biomarkers for their ability to stratify Stage I patients based on risk for recurrent disease. The most promising biomarkers will be used to develop a classification algorithm for prognosticating recurrent disease.

Aim 3: Independent validation of the optimal combination of biomarkers against serum specimens we obtained through collaboration with the Cancer and Leukemia Group B (CALGB). This validation study, CALGB 150809, is a fully IRB-approved companion study to CALGB 140202 and will test the biomarker panel developed as the objective of this proposal with a multi-institutional cohort of specimens (n=230).

A variety of molecular approaches have been explored as a means to stratify Stage I NSCLC patients based on clinical outcome (disease-free recurrence). The key studies in this area have primarily focused on the associations of expression microarray data from the primary tumor with locoregional lymph node status or outcome with the ultimate goal of identifying a prognostic, multi-gene signature to select NSCLC patients for systemic adjuvant chemotherapy.

All serum/plasma specimens were collected immediately prior to tumor resection with full IRB approval and written (individual) patient consent. Other collection protocols and storage conditions have been defined elsewhere. Groupings for the serum specimens meeting the inclusion criteria outlined below are provided in Table 9.

TABLE 9
Basic characteristics of groups defined
for biomarker discovery/validation.

| | | | Gender | |
|---|---|---|---|---|
| Histological Groups | NM* | Recurrence** | Male | Female |
| adenocarcinoma | ₁₋₂N₀M₀ | No | 44 | 0 |
| Squamous Cell Carcinoma | ₁₋₂N₀M₀ | No | 26 | 9 |
| Adenocarcinoma | ₁₋₂N₀M₀ | Yes | 13 | 4 |
| Squamous Cell Carcinoma | ₁₋₂N₀M₀ | Yes | 11 | |
| Adenocarcinoma | ₁₋₂N₁₋₂M₀ | N/A | 38 | 2 |
| Squamous Cell Carcinoma | ₁₋₂N₁₋₂M₀ | N/A | 22 | 0 |

*Based on pathological staging;
**see inclusion criteria.
^{‡} median follow up is 3.4 years for entire cohort.

All specimens were collected prospectively at RUMC with ≧2 years clinical follow-up data available. All patients will be pathologically staged, with standard hilar and mediastinal lymph node dissections. Hematoxylin and eosin staining was performed to identify the presence of metastatic disease. Patients with no pathological evidence of metastatic progression upon anatomic resection and having no disease recurrence within 2-years of resection will be considered to have “no recurrence”. All specimens will have sufficient serum available to complete objectives. All patients will be naïve to either chemo- or radiotherapy.

The general approach proposed for the serum biomarker discovery will minor that used by Bijian et al. for the identification of circulating biomarkers for oral squamous cell carcinoma. Briefly, they used 2-dimensional HPLC of pooled, iTRAQ-labeled serum specimen groups (control vs. cancer-bearing; invasive vs. non-invasive tumors) followed by online ion trap mass spectrometry to identify and quantify a range (1,100) of differentially expressed candidate biomarkers (p<0.05) between their study groups.

High-abundance Protein Depletion—Serum proteomics is generally thought to be a technically challenging undertaking given the broad range of proteins concentrations (10-12 orders of magnitude) normally present in serum/plasma specimens. This large dynamic range makes the detection of low and ultra-low abundance proteins difficult in unfractionated specimens. To obviate this issue we currently use the Agilent MARS Hu-14 column to remove 90-95% of the 14 most abundant proteins in serum specimens to improve access to the lower-abundance proteins for biomarker discovery. [Note: we recognize that depletion of these proteins may, in fact, slightly reduce the number of identified proteins due to no-specific co-depletion. For this reason our depletion protocol ensures that the eluate from the HU-14 column are archived for future analysis]

The high-abundance proteins may be depleted from 100 pre-surgical serum specimens (40 μL each) that represent the 6 groups outlined above (n=10 per group; except groups 3 & 4 where n=5) using the Agilent MARS HU-14 column (4.6×100 mm), according to manufacturer protocols. Specimens will be randomly selected by our statistician (Dr. Basu) for this study from our complete repository holdings (as outlined in Table I) for each group and will be matched as much as possible across these histological groups for common demographic categories (e.g. age, gender, etc.). All experiments will be performed blinded and in non-redundant replicates to limit sampling bias. Specimen depletion will be accomplished with technical replicates and employ our Shimadzu Prominence HPLC system. The resulting low-abundance protein fraction (estimated: 240-340 μgs total) will be acetone precipitated to both concentrate and desalt protein mixtures from the depletion buffers.

Trypsinization and iTRAQ labeling: All individual serum specimens depleted of the high-abundance proteins will be prepared for the MS analysis using protocols recommended by Applied Biosystems for iTRAQ analyses, with minimal modifications. Complete specimen digestion with sequencing-grade trypsin (Promega Corp.) will then be accomplished using manufacturer's defined protocols. Each tryptic digest will be labeled with a unique iTRAQ reagent according to the 6 groups defined in the patient cohorts section; namely 113-119 Da. An additional group of peptide reference standards (Sigma-Aldrich Corporation will also be labeled (121 Da) to serve as a control to control inter-run QC/QA and ensure batch-to-batch reproducibility.

Multi-dimensional Protein Identification (MudPIT) experiments: Specimens prepared above will be fractionated for mass spectral studies using an online (directly infused) multi-dimensional HPLC strategy (also known as the “MudPIT”). Approximately 20-50 μgs of each iTRAQ-labeled peptide mixtures will be processed in each chromatographic run. The first chromatographic fractionation will be accomplished via strong cationic exchange using a 10-step volatile salt gradient. All eluted peptides will be trapped for resolution in a second chromatographic dimension using a reversed-phase peptide cartridge. Upon valve switching, a second trapping cartridge will then be loaded with the next SCX gradient fraction while the trapped SCX fraction will be resolved on a reversed phase column and infused via nanoESI-into our Thermo LTQ XL linear ion trap mass spectrometer for analysis. Resolved, iTRAQ-labeled peptides will be analyzed in data-dependant mode with MS/MS scans for the 4-10 most abundant peptides (ion threshold of 500 counts) from each MS scan. iTRAQ label signature masses (i.e. 113-119, and 121 Da) will be monitored using pulsed induced dissociation (PQD) to obtain the proper quantification results of the identical iTRAQ-labeled peptides. All runs will be performed in technical replicates to ensure reproducibility of all data generated and the instrument tuned immediately before, and every 5th, analysis. Specific peptides identified in the iTRAQ experiment will be subjected to multiple reactions monitoring (MRM) for peptide quantity and protein identity confirmation studies. Parent peptide masses and the daughter fragmentation patterns will be obtained from the MASCOT results file. This database will be used to generate the MRM values for this set of experiments. iTRAQ label signature masses will be monitored using PQD to obtain the proper quantification results of the identical iTRAQ-labeled peptides. Furthermore, additional peptides corresponding to the identified proteins, but not observed in the discovery phase, will also be specifically monitored to provide additional confidence in the initial findings. If additional resolution is needed for this analysis, a Thermo LTQ FT-ICR is available at the UIC Mass Spectrometry Facility. Extensive consideration was put into the experimental design for this study using the suggestions of Oberg and Vitek to minimize potential sources of experimental bias and promote our ability to detect true quantitative changes between the groups tested.

Bioinformatics (Protein identification): Data analysis (protein identification and relative peptide quatitation) will be carried out using the Mascot (Matrix Science), and Bioworks 3.3.1 (Thermo) platforms, similar to methods we previously reported. Raw data will be extracted from the MS data files using the data extractor module in the Bioworks software package and then subjected to protein library search by the Mascot and Sequest algorithms for protein identification. Protein database searching will be restricted to tryptic peptides within the human database and MS data of indentified proteins will be subjected to decoy data base search (with all false positive proteins rejected). Precursor mass error of data obtained from LTQ-FT will be 10 ppm and 0.5 Da for the LTQ-XL. The complete list of identified peptides will then be transferred to a Microsoft Access (Microsoft, Redmond, Wash.) database for grouping of results into proteins and calculation of ratios and standard deviation. Confidence of protein identification will be selected according to a 95% confidence and a minimum of 30% sequence coverage and no less than two peptides identified per protein. Alternately, the University of Illinois at Chicago Mass Spectrometry Facility routinely performs this analysis using Scaffold v3.0 on a fee-per-project basis.

Analysis of Gene Expression Microarray Data—Data used for this subaim were obtained directly from the study investigators. All expression data sets were profiled using the Affymetrix Human U133A Gene Expression Microarray platform. The primary data sets (.CEL files) examined here were from Raponi, et al. (T1-4N0M0 n=83; T1-4N1-2M0 n=47) and Shedden et al. (T1-4N0M0 n=201; T1-4N1-2M0 n=90), and will be compared based on recurrence-free survival. The histological diagnoses were exclusively adenocarcinoma for the Shedden, whereas the Raponi data set was squamous cell carcinoma.

Probe level normalization of the .CEL files will be conducted by the RMA method. Batch effect from different laboratories will be evaluated using principal component analysis. If a batch effect is existed among the datasets, a Mean-Centering Method will be used to remove the batch effect as we previously accomplished. Briefly, the mean of each feature across all the samples within each batch is set to zero. This approach is also referred to as zero-mean, or one-way analysis of variance adjustment. It will be implemented in the pamr R package (http://cran.r-project.org/web/packages/pamr). After normalization and batch effect removal, all the datasets will be assembled to identify metastasis-associated genes. An un-paired student T-test will be used to identify differentiated genes between non-metastasis and metastasis samples. A cut off p-value≦0.05 with 2 fold change as cut off to find significantly differentially-expressed genes.

Biomarker Selection Methodology—Candidate biomarker selection will be conducted using the methods defined below. Briefly, proteins identified in our proteomics section that were modulated more than 1.5 fold in the patients with disease recurrence within 2 years of resection (relative to no recurrence) be filtered and intersected with the analyzed expression array data. We anticipate approximately 200-500 proteins (or targets) will be in this category and will represent the tumor secretome. These targets will then be categorized into functional categories according to Gene Ontology (GO) definitions using The Database for Annotation, Visualization and Integrated Discovery (DAVID) and GOfetcher tools. A Gene Ontology functional term enrichment p value less than 0.05 was considered significant. In parallel to this effort, we will also perform this pathway analysis using the Ingenuity canonical pathways analysis tool. Similar to GO analysis, a pathway with an enrichment p value less than 0.05 was considered to be a significantly regulated pathway (Ingenuity Systems, Redwood City, Calif.). We will then use support vector machine recursive feature elimination (SVM-RFE) as the primary method to filter out the optimum targets (candidate biomarkers) by using SVM in a wrapper-style. The algorithm selects a subset of features for a particular learning task. The basic algorithm is the following: 1) Initialize the data set to contain all features, 2) Train an SVM on the data set, 3) Rank features according to ci=(wi), 4) Eliminate the lower-ranked 50% of the features, 5) Return to step 2. At each RFE step 4, a number of genes are discarded from the active variables of an SVM classification model. A minimum of 10 Gene Ontology (GO) definitions will be represented, and ranked based on significance for internal validation. Potential pitfalls and alternative strategies—Currently, our overall operation is geared towards the analysis of serum specimen, so this was our natural preference. In general, investigators weary of serum-based proteomics are concerned over the potential loss of biomarkers to proteolysis during clot formation. To obviate this problem we have strict protocols relating to the manner in which serum is processed to minimize variation from serological methods and maintain reproducibility. However, relevant differences in serum proteins whether intact or partially degraded will be detected by our general strategy and allow our objectives to be met. Quantitative Luminex Assay Development and Characterization—Luminex assay development: Whenever possible, commercially-available immunobead assays built on the Luminex platform will be used for the described studies. Only when a commercial source of validated assays for a selected candidate biomarker is not available will custom assays be developed. Development of the custom sandwich immunobead assays for each candidate biomarker will employ antibody pairs (monoclonal capture/polyclonal detection) purchased through a commercial antibody distributor (such as R&D Systems). We do not propose to develop or contract the development of any immunoreagent for this study and will consider the availability of immunoreagents as a factor in our candidate biomarker selection algorithms.

Methods for custom assay development will be consistent with those we defined previously and recommended by the Luminex Corporation. Following successful individual assay development and performance characteristics determination (see below), multiplex assays containing 5-10 analyte assays per panel will be constructed. Assay groupings will be based on having low protein sequence homology across the group as to avoid potential issues with cross-reactivity. Validation of these multiplex assays for cross-reactivity will be performed using a modified ‘leave one out’ protocol from Luminex Corporation. A 10% difference in median fluorescence intensities (MFI) values will be our threshold for inter-assay interference for each individual target. Performance characteristics will be re-established for all individual assays in multiplex groups.

Guidelines to ensure assay performance (Quality Assurance): Technical and clinical validation of all assays will require determination of the following parameters: inter- and intra-assay precision, QC monitoring, assay reproducibility, assay linearity, recombinant antigen batch reproducibility, and assay standard (polyclonal antibody) batch reproducibility.

Internal Validation and Biomarker Panel Development—Evaluation of the candidate biomarkers with individual patient specimens will proceed consistent with methods we have already reported. A total of 309 NSCLC evaluable serum specimens from 3 patient populations (mixed histology/gender) will be included in this study and are defined in Table I. The groups consisted of 99 patients with early-stage (T1-3N0M0) NSCLC with no recurrence and 28 patients with early-stage (T1-3N0M0) NSCLC and have pathologically-confirmed disease within 2 years of the initial resection. We will also test our panel patients with positive lymph node (T1-3N1-2M0) disease (n=82). Specimens used for this study will be non-redundant from the discovery efforts of Aim 1. Details regarding patient inclusion criteria and collection protocols have been previously reported by our group. This cohort has a median follow up of 3.4 years. Univariate analysis of all tested biomarkers will be conducted, as previously defined, as will the multivariate panel selection algorithms (Random Forest and RCART). The findings from this aim will be validated. Multi-institutional Cohort Validation of the Biomarker Panel—Power calculation for validation studies—The primary objective of this aim is to assess the accuracy of our multivariate biomarker panel for correctly stratifying early-stage NSCLC patients according to 2-year recurrence-free survival.

Validation methodology for performance of the Luminex assays will approximate that used for Aim 2, with exception to the potential for running the panel as a single, multiplex panel (instead of individually). Development and validation of this panel will used the identical ‘leave-one-out’ validation protocol described in the methods for Aim 2. We anticipate that approximately 20 μL of the 100 μL aliquot obtained from the CALGB for validation purposes, providing the potential to test multiple panels/classification algorithms. An allowance to accomplish this comparative study has been made in the budget.

Methods of differentiating NSCLC from other non lung cancer disorders in a patient comprise the step of detecting the level of expression in a tissue sample of one or more genes from Table 6; wherein differential expression of the genes in Table 6 is indicative of lung cancer such as NSCLC rather than another disorder.

Methods of screening for an agent capable of modulating the onset or progression of lung cancer such as NSCLC, comprise the steps of exposing a cell to the agent; and detecting the expression level of two or more genes from Table 6.

Any of the disclosed methods described above may include the detection of at least two genes from the tables. In certain embodiments, the methods may detect all or nearly all of the genes in the tables. In some embodiments, one or more genes may be selected from a group consisting of the genes listed in Table 6.

Compositions are disclosed that comprise at least two oligonucleotides, wherein each of the oligonucleotides comprises a sequence that specifically hybridizes to a gene in Table 3 as well as solid supports comprising at least two probes, wherein each of the probes comprises a sequence that specifically hybridizes to a gene in Table 6.

Computer systems are also disclosed that comprise a database containing information identifying the expression level in lung tissue, serum or sputum of a set of genes comprising at least two genes in Table 6; and a user interface to view the information. The database may further include sequence information for the genes, information identifying the expression level for the set of genes in normal lung tissue and malignant tissue (metastatic and nonmetastatic) and may contain links to external databases such as GenBank; the databases maintained by the National Center for Biotechnology Information or NCBI (ncbi.nlm nih.gov/Entrez/). Other external databases that may be used include those provided by Chemical Abstracts Service (stnweb.cas.org/) and Incyte Genomics (incyte.com/sequence/index.shtml).

Kits useful for the practice of one or more of the disclosed methods are also disclosed. In some embodiments, a kit may contain one or more solid supports having attached thereto one or more oligonucleotides. The solid support may be a high-density oligonucleotide array. Kits may further comprise one or more reagents for use with the arrays, one or more signal detection and/or array-processing instruments, one or more gene expression databases and one or more analysis and database management software packages.

Methods of using the databases are disclosed, such as methods of using the disclosed computer systems to present information identifying the expression level in a tissue or cell of at least one gene in Table 6 comprising the step of comparing the expression level of at least one gene in Table 6 in the tissue or cell to the level of expression of the gene in the database.

Disclosed compositions and methods for detecting the level of expression of genes that may be differentially expressed dependent upon the state of the cell, i.e., normal versus cancerous. As used herein, the phrase “detecting the level expression” includes methods known in the art that quantitatively determine expression levels as well as methods that determine whether a gene of interest is expressed at all. Thus, an assay which provides a yes or no result without necessarily providing quantification of an amount of expression is an assay that requires “detecting the level of expression” as that phrase is used herein.

Although this disclosure describes preferred and alternate embodiments that will be used for the detection of NSCLC versus non-malignant disease and for the selection of treatment for patients with lung cancer, it is contemplated that the disclosed embodiments can be used in the detection of other cancers and more particularly other lung cancers. Thus, the instant disclosure should not be read to limit the use of the disclosed embodiments to NSCLC. Furthermore, the organization and type of the individual elements of the assays described herein represent preferred embodiments and should not be read to limit the use of alternate configurations and types. One of ordinary skill in the art can discern, from the description, alternate embodiments that can be contemplated by the designers.

1. A method of assessing non-small cell lung cancer (NSCLC) of a mammal, the method comprising:
collecting a biological sample from the mammal; and
measuring whether the mammal has an expression of at least one of a nucleic acid, at least one polypeptide encoded by said nucleic acid and at least one autoantibody to said polypeptide;
wherein a presence or absence of said nucleic acid, said polypeptide encoded by said nucleic acid and said autoantibody to said peptide is indicative that the mammal has NSCLC.

2. The method of claim 1 further comprising:
using the presence or absence of said nucleic acid, said polypeptide encoded by said nucleic acid and said autoantibody to said peptide to determine a treatment for NSCLC.

3. The method of claim 1 wherein said nucleic acid, said polypeptide encoded by said nucleic acid and said autoantibody to said polypeptide are selected from the group consisting of TNF-α, CYFRA 21.1, IL-1ra, IL-6, IFN-γ, IL-2Rα, CA125, MCP-1, CRP, MMP-2 and sE-selectin.

4. The method of claim 1 wherein the biological sample is selected from the group consisting of lung tissue, sputum and blood.

5. The method of claim 1 wherein the measuring further comprises:
detecting an elevated level of one or more of said nucleic acid, said polypeptide encoded with said nucleic acid and said autoantibody to said polypeptide in the biological sample, and
wherein said nucleic acid, said polypeptide encoded with said nucleic acid and said autoantibody to said polypeptide are selected from the group consisting of TNF-α, CYFRA 21.1, IL-1ra, MCP-1, MMP-2 and sE-selectin.

6. The method of claim 1 wherein the measuring comprises:
determining a presence or an absence of one or more of said nucleic acid, said polypeptide encoded with said nucleic acid and said autoantibody to said polypeptide in the biological sample,
wherein said nucleic acid, said polypeptide encoded with said nucleic acid and said autoantibody to said polypeptide are selected from the group consisting of TNF-α, CYFRA 21.1, IL-1ra, MCP-1, MMP-2 and sE-selectin, and
the method further comprises selecting a treatment for NSCLC based on the presence or absence of said nucleic acid, said polypeptide encoded with said nucleic acid and said autoantibody to said polypeptide in the biological sample.

7. The method of claim 1 further comprising detecting elevated levels of a plurality of nucleic acids simultaneously using a nucleic acid array.

8. The method of claim 1 further comprising detecting elevated levels of a plurality of polypeptides simultaneously using a polypeptide array.

9. The method of claim 1 further comprising using the presence or absence of said nucleic acid, said polypeptide encoded by said nucleic acid and said autoantibody to said peptide to assess NSCLC risk and determining whether the mammal requires further screening with computed tomography (CT).

10. The method of claim 1 wherein the level can be determined using at least one of PCR, in situ hybridization or immunohistochemistry.

11. The method of claim 1 further comprising:
determining whether or not the mammal has lung cancer versus a nonmalignant disease by detecting an elevated level of said nucleic acid, said polypeptide encoded with said nucleic acid and said autoantibody to said polypeptide is selected from the group consisting of IMPDH, phosphoglycerate mutase, ubiquillin, annexin I, annexin II, and heat shock protein 70-9B (HSP70-9B).

12. The method of claim 1 further comprising:
determining whether or not a mammal having lung cancer versus a nonmalignant disease by determining the level of said nucleic acid, said polypeptide encode with said nucleic acid and said autoantibody to said polypeptide is selected from the group consisting of inosine-5-monophosphate dehydrogenase (IMPDH), fumarate hydratase (FH), α-enolase, endoplasmic reticulum protein 29 (Erp29), annexin I, hydrosteroid 17-[3 dehydrogenase, methylthioadenosine phosphorylase (MTAP), annexin II, ubiquilin, c-Myc, NY-ESO, 3-oxoacid CoA transferase, p53 phosphoglycerate mutase, and heat shock protein 70-9B (HSP70-9B)]; and
using the presence or absence of an elevated level to determine treatment for a lung cancer such as NSCLC.
